(19) United States
(12) Patent Application Publication
(10) Pub. No.: US 2026/0335024 A1
(43) Pub. Date: Oct. 29, 2026

(54) GUIDED PROSTATE TREATMENT AND DISPLAY

(71) Applicant: Teleflex Life Sciences LLC, Wilmington, DE (US)

(72) Inventors: Matt Spottedhawk Ashley, Bend, OR (US); Avshalom Shenhav, Haifa (IL); Dina Sifri, Ramot Menashe (IL); Baruch Sabbah, Haifa (IL); Brian Wilkins, Pleasanton, CA (US); Ramesh Marrey, Mission Viejo, CA (US); Dean Hu, San Leandro, CA (US)

(73) Assignee: Teleflex Life Sciences LLC, Wilmington, DE (US)

(21) Appl. No.: 19/657,439

(22) Filed: Apr. 24, 2026

Related U.S. Application Data
(60) Provisional application No. 63/794,784, filed on Apr. 25, 2025.

(51) Int. Cl. A61B 34/20; A61B 8/00; A61B 8/12; A61B 17/00; A61B 17/04; A61B 34/10

(52) U.S. Cl. CPC A61B 34/20; A61B 8/12; A61B 8/4416; A61B 8/463; A61B 17/0469; A61B 2017/00274; A61B 2017/0034; A61B 2017/0409; A61B 2017/0419; A61B 2017/0464; A61B 2017/047; A61B 2034/105; A61B 2034/2048; A61B 2034/2051

(57) ABSTRACT

A delivery device is configured to deploy one or more implants to a prostate gland of a patient. The delivery device includes a handle assembly and an elongate shaft assembly; and a sensor coupled to the handle assembly and configured to locate a portion of the elongate shaft assembly with respect to anatomy of the patient.

### CROSS REFERENCE TO RELATED APPLICATIONS

This application claims the benefit of priority under 35 U.S.C. § 119(e) and 37 C.F.R. § 1.78 to provisional Application no. 63/794,784 filed on Apr. 25, 2025, titled “GUIDED PROSTATE TREATMENT AND DISPLAY” which is hereby incorporated by reference herein in its entirety.

### FIELD

The subject matter of this patent document relates to the field of medical devices. More particularly, but not by way of limitation, the subject matter relates to medical devices, systems, and methods for relieving pressure on a prostatic urethra by compressing at least a portion of a prostate gland.

### BACKGROUND

Benign Prostatic Hyperplasia (“BPH”) is one of the most common medical conditions that affect men, especially elderly men. It has been reported that, in the United States, more than half of all men have histopathologic evidence of BPH by age 60 and, by age 85, approximately 9 out of 10 men suffer from the condition. Moreover, the incidence and prevalence of BPH are expected to increase as the average age of the population in developed countries increases.

The prostate gland enlarges throughout the life of a man. In some men, the prostatic capsule around the prostate gland may prevent the prostate gland from enlarging further. This causes the inner end of the prostate gland to squeeze the urethra. This pressure on the urethra increases resistance to urine flow through the end of the urethra enclosed by the prostate. Thus, the urinary bladder has to exert more pressure to force urine through the increased resistance of the urethra. Chronic over-exertion causes the muscular walls of the urinary bladder to remodel and become stiffer. This combination of increased urethral resistance to urine flow and stiffness and hypertrophy of urinary bladder walls leads to a variety of lower urinary tract symptoms (LUTS) that may severely reduce the quality of life of the patient. These symptoms include weak or intermittent urine flow while urinating, straining when urinating, hesitation before urine flow starts, feeling that the bladder has not emptied completely even after urination, dribbling at the end of urination or leakage afterward, increased frequency of urination particularly at night, urgent need to urinate etc.

In addition to patients with BPH, LUTS may also be present in patients with prostate cancer, prostate infections, and chronic use of certain medications (e.g., ephedrine, pseudoephedrine, phenylpropanolamine, antihistamines such as diphenhydramine, chlorpheniramine etc.) that cause urinary retention especially in men with prostate enlargement.

Despite extensive efforts in both the medical device and pharmacotherapeutic fields, current treatments for BPH remain only partially effective and are burdened with significant side effects. Thus, there remains a need for the development of new devices, systems and methods for treating BPH as well as other conditions in which one tissue or anatomical structure impinges upon or compresses another tissue or anatomical structure.

### BRIEF SUMMARY

In one embodiment a method of treating a prostate gland includes: generating a plurality of images of an anatomy of a patient; based on the plurality of images, generating an anatomical model of the anatomy; inserting, into the patient, an implant delivery device, wherein the implant delivery device includes at least one spatial sensor; aligning the implant delivery device with the anatomy based on the anatomical model and data received from the at least one spatial sensor; tracking at least one of a position, a location, or an orientation of at least a portion of the implant delivery device based on the data; placing an implant in the anatomy via the implant delivery device.

In one embodiment, a delivery device is configured to deploy one or more implants to a prostate gland of a patient. The delivery device includes a handle assembly and an elongate shaft assembly; and a sensor coupled to the handle assembly and configured to locate a portion of the elongate shaft assembly with respect to anatomy of the patient.

In one embodiment, a system for delivering and deploying implants to a prostate gland of a patient, includes an implant delivery device including an elongate shaft assembly; a spatial sensor coupled to the implant delivery device and configured to provide data on the position and/or orientation of the elongate shaft assembly relative to the anatomy of the patient; a processing unit configured to generate an anatomical model of the anatomy from preoperative imaging data; a display configured to provide intraoperative guidance by integrating the spatial sensor data with the anatomical model.

In one embodiment, a method of treating a prostate gland includes: generating a plurality of images of an anatomy of a patient; based on the plurality of images, generating an anatomical model of the anatomy; inserting, into the patient, an elongate member or portion of an implant delivery device, wherein the implant delivery device includes at least one spatial sensor; aligning the elongate member or portion of the implant delivery device with the anatomy based on the anatomical model and data received from the at least one spatial sensor; tracking at least one of a position, a location, or an orientation of at least a portion of the implant delivery device based on the data; and placing an implant in the anatomy via the implant delivery device. This embodiment enables generation of high-resolution cross-sectional images that can be integrated and assembled into a three-dimensional representation, providing comprehensive volumetric data of the patient's anatomy for accurate treatment planning.

Optionally, in some embodiments, the plurality of images includes at least one image selected from the group consisting of MRI images, CT images, and transrectal ultrasound images. This embodiment enables generation of high-resolution cross-sectional images that can be integrated and assembled into a three-dimensional representation, providing comprehensive volumetric data of the patient's anatomy for accurate treatment planning.

Optionally, in some embodiments, the anatomical model includes one or more representations of anatomical structures including the prostate gland and/or the prostatic urethra, and identifies potential sites for implant placement. This embodiment facilitates pre-surgical planning by isolating specific structures or regions of interest and identifying optimal implant locations, thereby improving the precision of implant deployment.

Optionally, in some embodiments, the anatomical model is generated using a machine learning algorithm that enhances image segmentation and identification of anatomical landmarks. This embodiment improves the accuracy of anatomical structure differentiation by interpreting grayscale values or signals corresponding to different tissue densities or properties, enabling creation of a more detailed and interactive anatomical model compared to other techniques.

Optionally, in some embodiments, aligning the implant delivery device includes translationally and/or rotationally positioning the elongate member or portion of the implant delivery device relative to a reference portion of the anatomy. This embodiment enables precise calibration of the delivery device position and orientation, reducing accumulated error over time and improving the accuracy of subsequent tracking during the procedure compared to other techniques.

Optionally, in some embodiments, the reference portion is a bladder neck. This embodiment provides a consistent and identifiable anatomical landmark for zeroing, resetting, or calibrating the location, position, and/or orientation of the implant delivery device, enabling reliable alignment with the anatomical model.

Optionally, in some embodiments, the aligning step includes using data from the spatial sensor to assist in the alignment. This embodiment enables real-time feedback during the alignment process, allowing the clinician to make precise adjustments and orient the implant delivery device to the anatomical model with improved accuracy compared to other techniques.

Optionally, in some embodiments, the method further includes generating real-time intraoperative guidance updates based on the data from the at least one spatial sensor to aid in accurate implant navigation and placement. This embodiment enables the clinician to adjust for movements or changes in the anatomy of the patient, differences discovered between the anatomical model and the actual anatomy, or failure of an implant to be properly placed, thereby improving patient outcomes compared to other techniques.

Optionally, in some embodiments, the intraoperative guidance updates include visualization outputs that display real-time movement of the elongate member or portion of the implant delivery device in conjunction with the anatomical model. This embodiment provides visual indicators such as distance measurements, angle readouts, or graphical representations of the elongate member position relative to anatomical landmarks, enhancing situational awareness for the clinician during the procedure.

Optionally, in some embodiments, the tracking of the implant delivery device is based on a combination of accelerometer and gyroscope data from the spatial sensor. This embodiment enables comprehensive capture of both linear acceleration and rotational rates, providing motion data that can be processed to derive the current acceleration, velocity, position, and/or orientation of the implant delivery device.

Optionally, in some embodiments, the data includes six degrees of freedom. This embodiment enables the spatial sensor to comprehensively capture the translational and rotational dynamics of the implant delivery device in three-dimensional space, providing complete tracking of device movement during the procedure.

Optionally, in some embodiments, placing the implant includes deploying the implant at an angle relative to a longitudinal axis of the urethra to target a specified lobe of the prostate gland. This embodiment enables targeted treatment of specific lobes of the prostate gland, such as the left lateral lobe, right lateral lobe, or middle lobe, allowing for customized treatment based on the patient's anatomy.

Optionally, in some embodiments, the implant delivery device includes a sensor mounted to a shaft distal portion of the elongate member, the sensor configured to measure a distance from the shaft distal portion to a prostate capsule boundary. This embodiment provides real-time feedback to guide needle deployment depth and tissue compression during the procedure, enabling confirmation that the needle will successfully penetrate the prostate capsule boundary at an appropriate depth.

Optionally, in some embodiments, the sensor includes a Type-A ultrasound transducer configured to emit ultrasound waves through prostatic tissue toward the prostate capsule boundary. This embodiment provides a simpler, smaller, and less costly solution than a Type-B ultrasound array, making it suitable for integration into a single-use delivery device while still providing the necessary distance measurement information for successful deployment.

Optionally, in some embodiments, the method further includes guiding a user to place the elongate member at a compression angle that compresses the prostate gland and optimizes a distance to the prostate capsule boundary prior to deploying a needle through the prostatic tissue. This embodiment assists the clinician in achieving proper tissue compression prior to needle deployment, optimizing the distance between the shaft distal portion and the prostate capsule boundary for deploying implants.

Optionally, in some embodiments, deploying the needle includes firing the needle at the compression angle to pierce the prostate capsule boundary while avoiding bone strike. This embodiment ensures proper penetration depth through the prostatic tissue to reach the outer surface of the prostatic capsule without extending beyond into surrounding anatomical structures such as pelvic bone, preventing failed deployments.

In one embodiment, a delivery device is configured to deploy one or more implants to a prostate gland of a patient. The delivery device includes a handle assembly and an elongate shaft assembly; and a sensor coupled to the handle assembly and configured to locate a portion of the elongate shaft assembly with respect to anatomy of the patient. This embodiment enables real-time tracking of the delivery device position relative to the patient's anatomy, improving the accuracy of implant placement compared to prior delivery devices that lack spatial sensing capabilities.

Optionally, in some embodiments, the sensor is an inertial measurement unit (IMU) that includes accelerometers and gyroscopes to provide data on linear and rotational movement of the elongate shaft assembly. This embodiment provides comprehensive data on movement, position, location, and/or orientation of the implant delivery device through measurement of specific force, angular rate, and real-time tracking of such data.

Optionally, in some embodiments, the delivery device further includes an ultrasound transducer mounted to a distal portion of the elongate shaft assembly and configured to measure a distance from the distal portion to a prostate capsule boundary. This embodiment provides tissue structure gradient information and distance measurements to the prostate capsule boundary prior to needle deployment, helping to properly and fully deploy the implant and avoid a bone strike.

Optionally, in some embodiments, the ultrasound transducer is a Type-A ultrasound transducer configured to determine the distance within a narrow or fixed vector corresponding to a needle path. This embodiment provides targeted distance measurement specific to the needle deployment vector, replacing the prior approach of relying on training guidance to compress the tissue with a certain degree of angulation applied to the handle, with real-time, localized feedback.

In one embodiment, a system for treating a prostate gland of the patient includes the delivery device; an imaging device configured to capture images of the anatomy of the patient; and a processing element configured to receive the images of the anatomy of the patient, and generate a three dimensional model of the anatomy. This embodiment enables creation of a detailed, interactive three-dimensional anatomical model that can be used for diagnostic purposes, pre-surgical planning, and intra-operative guidance, thereby improving the placement of implants and overall procedural outcome compared to other techniques.

Optionally, in some embodiments, the system further includes an inertial measurement unit (IMU) and a display, the display being configured to present real-time feedback via a user interface about the position and orientation of the elongate shaft assembly relative to the anatomy based on data from the IMU. This embodiment provides visual feedback to the clinician during the procedure, enabling iterative adjustments until the elongate member is correctly positioned for deployment of the implant.

Optionally, in some embodiments, the delivery device further includes an ultrasound transducer mounted to a distal portion of the elongate shaft assembly and configured to measure a distance to a prostate capsule boundary. This embodiment addresses a challenge in BPH treatment procedures of confirming that the needle will successfully penetrate the prostate capsule boundary at an appropriate depth, reducing the risk of failed deployments compared to other techniques.

In one embodiment, a system for delivering and deploying implants to a prostate gland of a patient includes an implant delivery device including an elongate shaft assembly; a spatial sensor coupled to the implant delivery device and configured to provide data on a position and/or orientation of the elongate shaft assembly relative to anatomy of the patient; a processing unit configured to generate an anatomical model of the anatomy from preoperative imaging data; and a display configured to provide intraoperative guidance by integrating the spatial sensor data with the anatomical model. This embodiment provides improved patient outcomes compared to prior methods and systems of treating BPH by enabling real-time tracking and guidance during the procedure through integration of spatial sensor data with preoperative anatomical modeling.

Optionally, in some embodiments, the spatial sensor is an inertial measurement unit (IMU) including accelerometers and gyroscopes to track linear and/or rotational movement of the elongate shaft assembly. This embodiment enables measurement of linear acceleration and rotational rates about orthogonal axes, providing six degrees of freedom for comprehensive tracking of the delivery device trajectory, attitude, position, location, and/or orientation.

Optionally, in some embodiments, the spatial sensor is an electromagnetic tracking device working in coordination with a field generator to track linear and/or rotational movement of the elongate shaft assembly. This embodiment enables precise monitoring of instrument positions and orientations relative to the patient's anatomy by detecting disturbances within a controlled electromagnetic field as the implant delivery device moves through the field.

Optionally, in some embodiments, the field generator creates a controlled electromagnetic field covering a surgical area. This embodiment provides a defined field range within which the spatial sensor can detect disturbances, enabling continuous updating and processing of data to provide a dynamic and accurate depiction of position, location, and/or orientation of surgical instruments in real-time.

Optionally, in some embodiments, the processing unit is configured to provide real-time intraoperative updates of the elongate shaft assembly position and/or orientation on the display. This embodiment enables the clinician to adjust for movements or changes in the anatomy during the procedure, such as changes in the urethral axis compared to the anatomical model, improving procedural accuracy compared to other techniques.

Optionally, in some embodiments, the display presents preoperative planning outputs and intraoperative guidance simultaneously. This embodiment enables the clinician to view both the planned implant locations and real-time device position in a unified interface, facilitating comparison between the treatment plan and actual procedure progress.

Optionally, in some embodiments, the system further includes a transrectal ultrasound unit that supplements anatomical visualization during the procedure. This embodiment provides live intraoperative imaging in addition to images captured by the image sensor, enabling real-time visualization of the prostate and surrounding structures during implant deployment.

Optionally, in some embodiments, the processing unit utilizes a machine learning algorithm to refine the anatomical model based on intraoperative data of previous cases. This embodiment enables continual improvement of the anatomical model, planning guidance, and intraoperative guidance through training data generated from prior procedures, providing predictive insights into alternative surgical strategies and outcomes.

Optionally, in some embodiments, the implant delivery device further includes an ultrasound transducer mounted to a distal portion of the elongate shaft assembly and configured to measure a distance from the distal portion to a prostate capsule boundary. This embodiment provides real-time, localized feedback specific to the needle deployment vector, replacing the prior approach of performing needle deployment without real-time capsule distance feedback.

Optionally, in some embodiments, the ultrasound transducer is a Type-A ultrasound transducer configured to emit ultrasound waves through prostatic tissue toward the prostate capsule boundary. This embodiment provides the necessary information for successful deployment without the complexity, cost, and workflow disruption associated with Type-B ultrasound arrays, while being suitable for integration into a single-use delivery device.

Optionally, in some embodiments, the display is configured to provide real-time visual feedback indicating a position and orientation of the elongate shaft assembly relative to a prostate capsule boundary and a target deployment site. This embodiment enables more responsive guidance during positioning adjustments compared to other techniques by reducing latency between sensor measurement and feedback generation, improving the accuracy of device placement.

Optionally, in some embodiments, the implant delivery device further includes a chip-on-tip imaging component mounted to the distal portion of the elongate shaft assembly and configured to provide visual feedback for navigating the implant delivery device to a target location within the prostate gland. This embodiment enables the device to be easily navigated to the proper location within the prostate gland by providing imaging capabilities that generate visual feedback to assist in device navigation and implant deployment.

Optionally, in some embodiments, the ultrasound transducer and the chip-on-tip imaging component are integrated on a single shaft to provide combined imaging capabilities during the procedure. This embodiment provides combined imaging by integrating ultrasound for tissue structure gradient and distance measurement with chip-on-tip imaging for visual navigation, enabling both proper device navigation and confirmation of appropriate needle deployment depth.

Optionally, in some embodiments, the chip-on-tip imaging component is configured to process image data locally at the distal portion of the elongate shaft assembly and provide real-time visual guidance during device navigation and implant deployment. This embodiment reduces latency between sensor measurement and feedback generation by processing data locally at the shaft distal portion, improving the accuracy of device placement by enabling more responsive guidance during positioning adjustments.

Optionally, in some embodiments, a location or arrangement of the ultrasound transducer on the distal portion of the elongate shaft assembly is configurable. This embodiment enables optimization of the ultrasound transducer position to accommodate different anatomical configurations and procedural requirements, providing flexibility in device design and deployment approach.

Optionally, in some embodiments, a location or arrangement of the ultrasound transducer and the chip-on-tip imaging component on the distal portion of the elongate shaft assembly is configurable. This embodiment enables optimization of both imaging components to accommodate different anatomical configurations and procedural requirements, providing flexibility in achieving combined imaging capabilities for various clinical scenarios.

### BRIEF DESCRIPTION OF THE DRAWINGS

FIG. 1A illustrates a cross-sectional view of the anatomy surrounding a prostate in a human subject.

FIG. 1B illustrates an enlarged cross-sectional view of the anatomy surrounding a prostate.

FIG. 2 shows a coronal section through the lower abdomen of a male human suffering from BPH showing a hypertrophied prostate gland treated with an embodiment of the device of the present disclosure.

FIG. 3A-FIG. 3G show the various steps of a method of treating a prostate gland using the implant(s) and devices shown and described herein.

FIG. 4 shows a perspective view of an example of an implant in accordance with embodiments disclosed herein, for example as shown in FIGS. 2-3G.

FIG. 5 shows a view of an example of an implant delivery device in accordance with embodiments disclosed herein.

FIG. 6 shows a view of another example of an implant delivery device in accordance with embodiments disclosed herein.

FIG. 7 shows a view of a distal end portion of an elongate portion of an implant delivery device in accordance with embodiments disclosed herein.

FIG. 8 shows a perspective view of a portion of the implant delivery device of FIG. 6 and/or FIG. 7.

FIG. 9 shows a schematic view of an example of a system for treating the prostate with the implant delivery device of FIG. 5 and/or FIG. 6.

FIG. 10 shows a schematic view of another example of a system for treating the prostate with the implant delivery device of FIG. 5 and/or FIG. 6.

FIG. 11 shows examples of pre-procedure planning and intra-procedure guidance outputs from the systems of FIG. 9 and/or FIG. 10.

FIG. 12 shows a coronal section through a hypertrophied prostate gland, in a first position, being treated with an embodiment of the implant delivery device of the systems of FIG. 9 and/or FIG. 10.

FIG. 13 shows a coronal section through a hypertrophied prostate gland, in a second position, being treated with an embodiment of the implant delivery device of the systems of FIG. 9 and/or FIG. 10.

FIG. 14 shows an example output of an embodiment of the systems of FIG. 9 and/or FIG. 10.

FIG. 15 shows an example output of an embodiment of the systems of FIG. 9 and/or FIG. 10.

FIG. 16 shows an example output of an embodiment of the systems of FIG. 9 and/or FIG. 10.

FIG. 17 shows an example of a pre-procedure planning output of the systems of FIG. 9 and/or FIG. 10.

FIG. 18 shows another example of a pre-procedure planning output of the systems of FIG. 9 and/or FIG. 10.

FIG. 19 is a flowchart of a method of providing a BPH treatment procedure according to the systems and devices disclosed herein.

FIG. 20 illustrates a side view of a shaft distal portion with a sensor for measuring distance to a prostate capsule boundary, according to an embodiment.

FIG. 21 illustrates a side view of the shaft distal portion of FIG. 20 positioned to guide compression angle optimization, according to an embodiment.

FIG. 22 illustrates a side view of the shaft distal portion of FIG. 20 with a needle deployed at an optimized compression angle, according to an embodiment.

FIG. 23 is a simplified block diagram of components of a computing system of the systems of FIG. 9 and FIG. 10.

The drawing figures are not necessarily to scale. Certain features and components may be shown exaggerated in scale or in schematic form and some details may not be shown in the interest of clarity and conciseness.

### DETAILED DESCRIPTION

The present devices and associated methods provide clinicians with means to treat an enlarged prostate, which may be a symptom of BPH, to alleviate its impingement on the adjacent prostatic urethra. Implants disclosed herein can be placed using a disclosed device pursuant to a method for compressing a prostate gland or portion thereof according to the following description.

As used herein, the terms “prostatic implant” and “implant” and “anchor assembly” and “retainer” may be used interchangeably. Each implant, once fully assembled, may include a distal anchor or capsular tab (hereinafter “CT”), a connector member, portion or suture (hereinafter “suture”), and a proximal anchor or urethral endpiece (hereinafter “UE”). The CT and suture may be provided together as a unitary component or assembly, with the CT attached, fixed, integrally formed with, or coupled to one end of the suture. The UE may be provided as a separate component that is attached to the CT/suture after deploying the CT/suture to the targeted tissue, for instance after the CT has emerged from the distal end of the delivery needle, at or beyond the outer surface of the prostatic capsule, and the suture has been implanted within the prostatic tissue. Embodiments include attaching the UE to the suture of a CT/suture assembly after tightening the CT against the outer capsular surface and tensioning the suture through the targeted tissue.

The terms “actuator” and “actuator member” may be used interchangeably in some embodiments disclosed herein. In some examples, an “actuator” or “actuator member” may include or be synonymous with a manually engageable portion, member, part, or component (such as a button, dial, switch, toggle, knob, lever, or trigger) configured to be actuated, for example via manually induced movement (such as a button press or lever sweep).

As used herein, the term “suture” may be used to represent a connector, connector member, connector portion, or elongate middle portion or member of an implant extending between a distal anchor and a urethral endpiece, through the prostatic tissue.

The term “member” may be used herein to represent a subcomponent or subassembly of a larger component or assembly, or the term may represent the larger component or assembly itself.

The terms “elongate member” and “shaft” and “shaft assembly” may refer to the same or similar components and may be used interchangeably herein. Embodiments of a shaft assembly may include components configured to access a treatment site within a prostatic urethra, deploy one or more implants, irrigate the treatment site, and/or visualize the procedure from within the patient. Examples of the shaft assembly may include a scope tube configured to accommodate an endoscopic instrument or scope assembly within a lumen of the scope tube. Examples of a shaft assembly may include one or more components of a needle assembly, a suture assembly, and/or a cutter assembly, non-limiting examples of which are disclosed in U.S. Pat. No. 11,298,115 and U.S. Patent Application Publication No. 2021/0378658, the entire contents of each of which are incorporated by reference herein.

The term “procedure” may refer to a medical treatment method used to compress at least a portion of an anatomical structure or tissue, including tissue of the prostate gland, which may be enlarged relative to a corresponding healthy tissue.

The term “user” may refer to a clinician, doctor, nurse, or medical professional performing a procedure described herein, which may involve the implantation of one or more implants within a targeted portion of prostatic tissue, which may be enlarged relative to normal prostatic tissue. In some examples, the term “user” may refer to more than one person, including two or more medical professionals working together to perform a procedure.

The terms “deploy” and “implant” may be used interchangeably herein, referring to the release and/or ejection of a disclosed implant or subassembly from a delivery device into a tissue being targeted. Accordingly, for instance, an implant may be fully deployed or implanted when the distal anchor of an implant is positioned at an outer surface of the prostatic capsule, the suture has been advanced through and tensioned within the targeted prostate lobe, and the UE has been attached to the tensioned suture at the urethral side of the lobe.

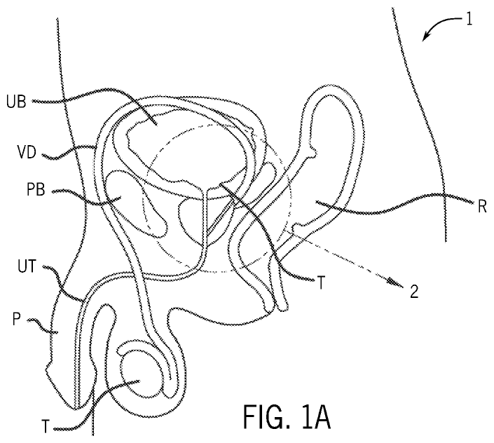

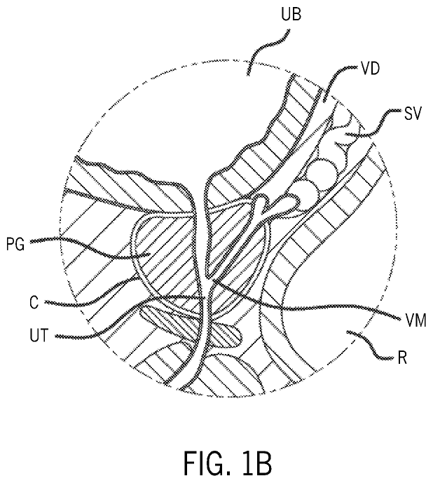

FIGS. 1A and 1B illustrate various features of the urological anatomy of a human subject. The prostate gland PG is a walnut-sized muscular gland located adjacent the urinary bladder UB. The urethra UT runs through the prostate gland PG. The prostate gland PG secretes fluid that protects and nourishes sperm. The prostate also contracts during sperm ejaculation to expel semen and provide a valve to keep urine out of the semen. A firm prostatic capsule PC surrounds the prostate gland PG.

The urinary bladder UB holds urine. The vas deferentia VD define ducts through which semen is carried, and the seminal vesicles SV secrete seminal fluid. The rectum R is the end segment of the large intestine through which waste is dispelled. The urethra UT carries both urine and semen out of the body. Thus, the urethra is connected to the urinary bladder UB and provides a passageway to the vas deferentia VD and seminal vesicles SV.

The trigone T is a smooth triangular end of the bladder. It is sensitive to expansion and signals the brain when the urinary bladder UB is full. The verumontanum VM is a crest in the wall of the urethra UT where the seminal ducts enter. The prostatic urethra is the section of the urethra UT that extends through the prostate.

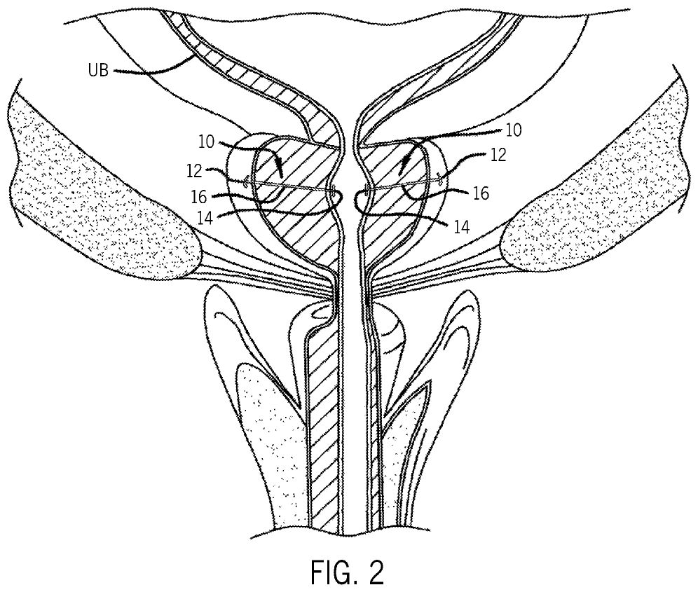

FIG. 2 shows a coronal section through the lower abdomen of a male human suffering from BPH showing a hypertrophied prostate gland treated with an embodiment of the device of the present disclosure. It has been discovered that the enlarged prostate gland is compressible and can be retracted so as to relieve the pressure from the urethra. In accordance with one embodiment of the present disclosure, a retaining device can be placed through the prostate gland in order to relieve the pressure on the urethra. In FIG. 2, a retainer or implant 10 is implanted in the prostate gland. Implant 10 comprises a distal anchor or capsular tab 12 (or “CT”) and a proximal anchor or urethra endpiece (or “UE”) 14. After assembly of the implant 10, the distal anchor 12 and proximal anchor 14 are connected by a middle portion or connector 16, which may comprise, include, or resemble a suture. The radial distance from the urethra to the distal anchor 12 is greater than the radial distance from the urethra to the proximal anchor 14. The distance or tension between the anchors is sufficient to compress, displace or change the orientation of an anatomical region between distal anchor 12 and proximal anchor 14. The connector 16 may be substantially inelastic so as to maintain a constant force or distance between the proximal and distal anchors, or it may be elastic to facilitate drawing the proximal and distal anchors closer together. In the embodiment shown in FIG. 2, distal anchor 12 is located on the outer surface of the capsule of prostate gland CP and acts as a capsular anchor. Alternatively, distal anchor 12 may be embedded inside the tissue of prostate gland PG, or in the surrounding structures around the prostate, such as periosteum of the pelvic bones, within the bones themselves, pelvic fascia, cooper's ligament, muscles traversing the pelvis or bladder wall. Also, in the embodiment shown in FIG. 2, proximal anchor 14 may be located on the inner wall of urethra UT, where it acts as a urethral anchor. Alternatively, proximal anchor 14 may be embedded inside the tissue of prostate gland PG or surrounding structures as outlined above. Distal anchor 12 and proximal anchor 14 are implanted in the anatomy such that a desired distance or tension is created in connector 16. This causes distal anchor 12 and proximal anchor 14 to retract or compress a region of prostate gland PG to relieve the urethral constriction. In FIG. 2, two implants 10 are implanted in a lateral lobe (side lobe) of prostate gland PG. The various methods and devices disclosed herein may be used to treat a single lobe or multiple lobes, including one or more lateral lobes and the median lobe of the prostate gland or other anatomical structures, by deploying one or more implants to the targeted tissue.

The implants may be deployed at particular angles relative to the axis of the urethra to target one or more lateral lobes and/or median lobe of the prostate gland. For example, implant 10 may be deployed between the 1 o'clock and 3 o'clock position relative to the axis of the urethra to target the left lateral lobe of the prostate gland. In another example, implant 10 may be deployed between the 9 o'clock and 11 o'clock position relative to the axis of the urethra to target the right lateral lobe of the prostate gland. In another example, implant 10 may be deployed between the 4 o'clock and 8 o'clock position relative to the axis of the urethra to target the middle lobe of the prostate gland.

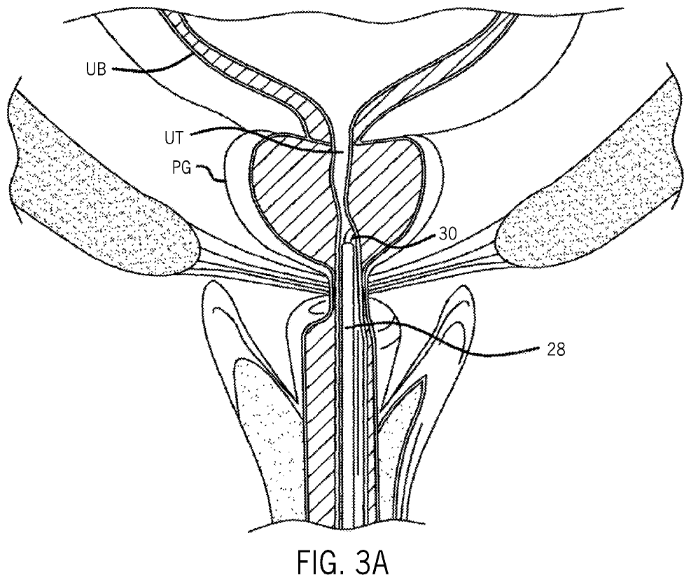

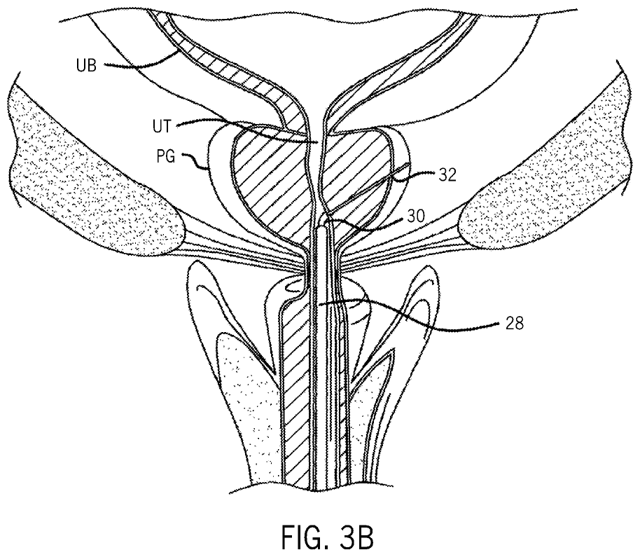

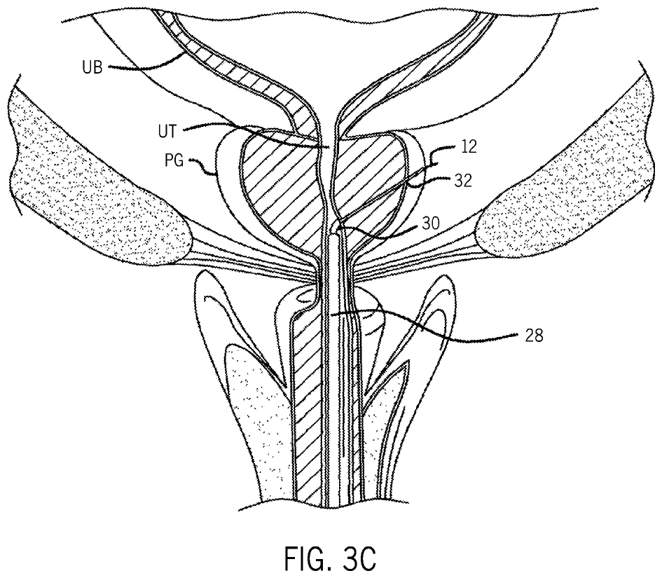

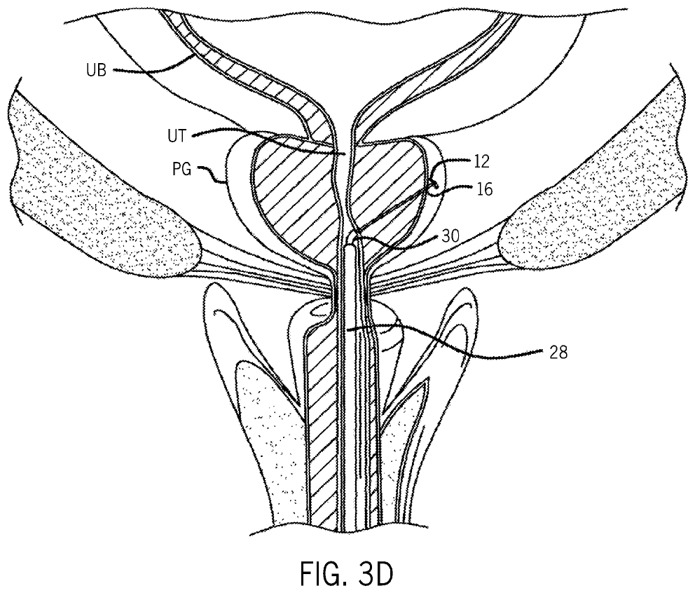

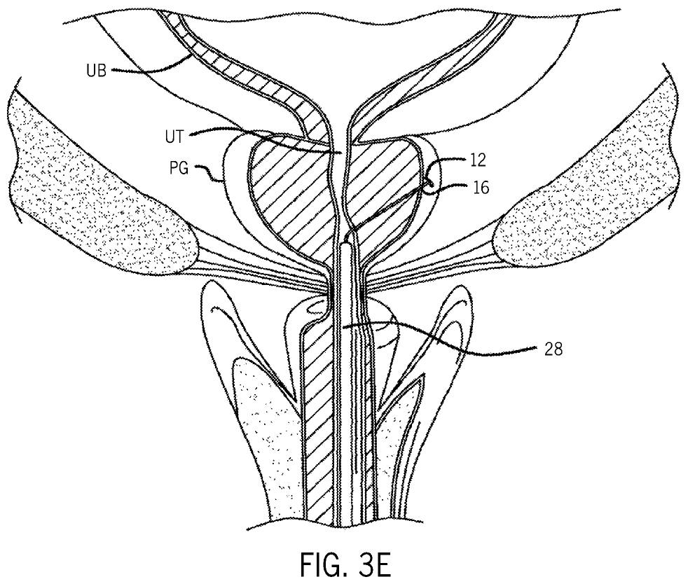

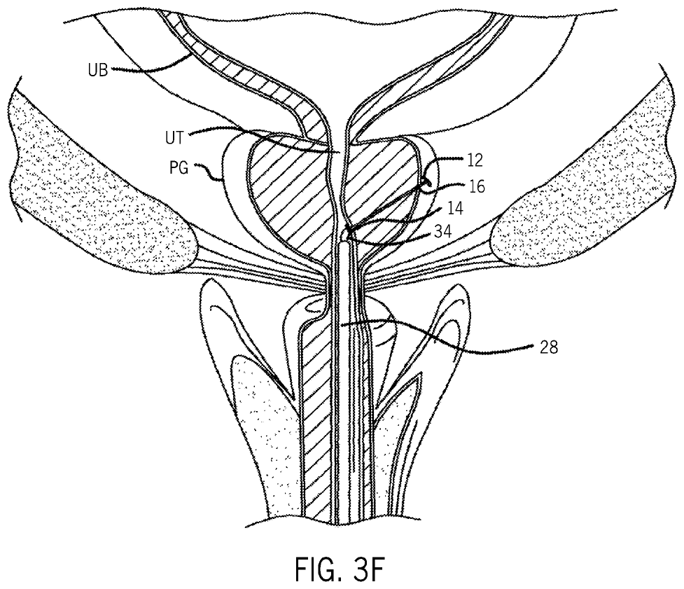

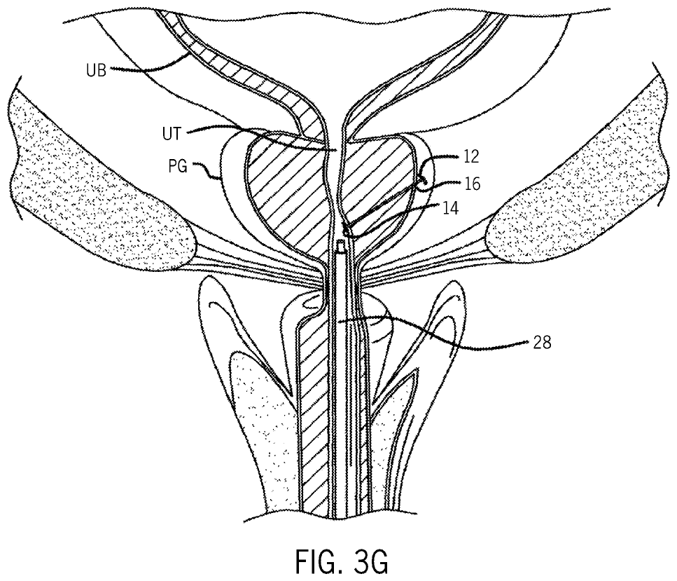

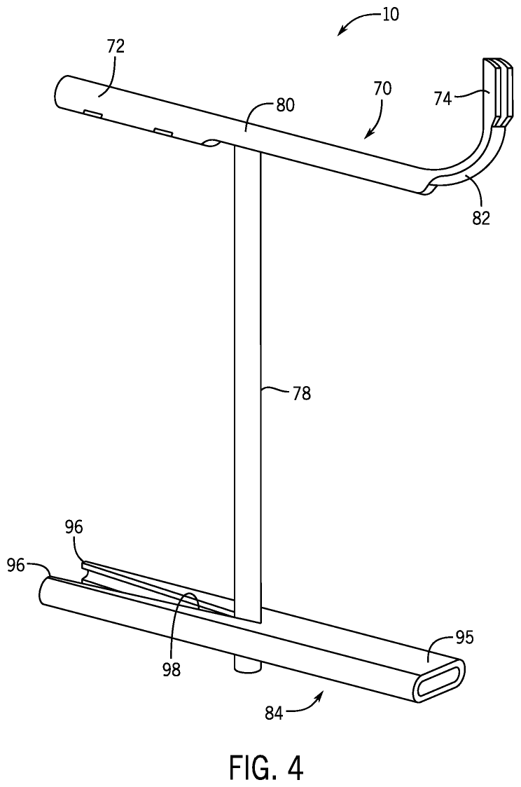

FIGS. 3A-3G show the various steps of a non-limiting example of a method of treating a prostate gland using one or more of the implants and associated delivery devices disclosed herein (see e.g., FIG. 4). One or more of the illustrated steps may be modified or excluded in accordance with certain embodiments disclosed herein, for example those advantageously not requiring a sheath. One or more of the depicted steps, shown for illustrative purposes, may be performed in a different manner depending on the particular delivery device(s) used, embodiments of which are disclosed herein. Similar methods may also be implemented to deploy retainers, implants, or compression devices in other anatomical structures.

In the step shown in FIG. 3A, a sheath 28, which may comprise a standard resectoscope sheath, may be introduced into the urethra (trans-urethrally). Sheath 28 may be advanced through urethra UT such that the distal end of sheath 28 is positioned near a region of urethra UT that is obstructed by a hypertrophied prostate gland PG. A distal anchor delivery device 30 featuring an elongate member or shaft may then be introduced through sheath 28. Distal anchor delivery device 30 may be placed in the sheath 28 after the distal end of sheath 28 is positioned near the region of the urethra UT that is obstructed, or the distal anchor delivery device 30 may be pre-loaded in the sheath 28 before positioning of the sheath 28. Distal anchor delivery device 30 may be advanced through sheath 28 such that the distal end of distal anchor delivery device 30 emerges out of the distal end of sheath 28. Distal anchor delivery device 30 may be oriented such that a working channel opening of distal anchor delivery device 30 points towards a lateral lobe of prostate gland PG.

In the step shown in FIG. 3B, a penetrating member or needle 32 may be introduced through distal anchor delivery device 30. Needle 32 may be placed in distal anchor delivery device after the distal anchor delivery device 30 is advanced through sheath 28, or the needle 32 can be pre-loaded in the distal anchor delivery device 30. In one non-limiting embodiment, the needle 32 is a 20 gauge needle. Needle 32 is advanced through distal anchor delivery device 30 such that it emerges through the working channel opening. Needle 32 may be further advanced until it penetrates through the tissue of prostate gland PG and the distal end of needle 32 emerges out of the capsule of prostate gland CP.

In the step shown in FIG. 3C, the distal anchor 12 connected to the connector 16 may be deployed at, or just beyond, the outer surface of the prostatic capsule. In some embodiments, the distal anchor 12 may be deployed by being extended beyond the distal end of the needle 32. In other embodiments, the distal anchor 12 may be deployed by being held in place by a pusher or connector while the needle 32 is retracted, thus exposing the distal anchor and unsheathing distal anchor 12 and connector 16 upon continued needle retraction. Distal anchor 12 can be pre-loaded within the needle 32 or it can be loaded in the needle 32 after the needle 32 has been advanced through distal anchor delivery device 30.

In the step shown in FIG. 3D, the needle 32 may be removed from the distal anchor delivery device 30 by pulling the needle 32 in the proximal direction. In the step shown in FIG. 3E, the distal anchor delivery device 30 may be removed from the sheath 28 by pulling distal anchor delivery device 30 in the proximal direction. Also, the connector 16 may be pulled to orient the distal anchor 12 approximately perpendicularly or otherwise transverse to the connector 16, against the outer surface of the prostatic capsule.

In the step shown in FIG. 3F, the connector 16 may be passed through the proximal anchor 14 located on a proximal anchor delivery device 34. The proximal anchor delivery device 34 may be advanced through the sheath 28 such that the distal end of proximal anchor delivery device 34 emerges out of the distal end of sheath 28. A desired tension may be introduced in connector 16 such that distal anchor 12 is pulled by connector 16 with a desired force. The proximal anchor can additionally or alternatively be visualized through an endoscope or under fluoroscopy and advanced along the connector 16 until the desired retraction of the tissue is achieved. In other embodiments, the proximal anchor may be a v-shaped or clothespin-shaped piece that may be forced, in some cases at high speed, onto the connector 16 to fixedly engage the connector.

In the step shown in FIG. 3G, the connector 16 is attached to proximal anchor 14. Proximal anchor 14 is also released from proximal anchor delivery device 34, thus deploying proximal anchor 14 and the implant 10 as a whole in the targeted anatomy. Proximal anchor delivery device 34 and sheath 28 may then be removed from the anatomy, retracting proximally through the urethra.

This method may be used to retract, lift, support, reposition or compress multiple regions or lobes of the prostate gland PG. In the method shown in FIGS. 3A-3G, the distal anchor 12 is deployed on the outer surface of the prostatic capsule. Thus, the distal anchor 12 acts as a capsular anchor. Alternatively, distal anchor 12 may be deployed inside the tissue of prostate gland PG or beyond the prostate as outlined previously. Similarly, in the method shown in FIGS. 3A-3G, the proximal anchor 14 may be deployed on the inner wall of urethra UT, acting as a urethral anchor. Alternatively, proximal anchor 14 may be deployed inside the tissue of prostate gland PG. In some examples, as noted above, one or more of the delivery components or devices may not be used or necessary, non-limiting examples of which may include sheath 28.

FIG. 4 provides a perspective view of a non-limiting example of an anchor assembly or implant 10 deployed using the systems, devices, and assemblies herein. In an unconstrained configuration, the distal anchor component 70 (or “CT”) may include a head portion 72 which may be generally orthogonally oriented with respect to a tail portion 74. While housed in the distal portion of a delivery needle and prior to deployment at a target area, the distal anchor component 70 may be constrained to a generally straight configuration, only subsequently assuming the unconstrained (i.e., orthogonally oriented) configuration upon deployment from the needle assembly.

In certain embodiments, the distal anchor component 70 may be formed from a nitinol base stock that is generally tubular and can be shape-set to include the orthogonally oriented configuration of the head portion 72 with respect to the tail portion 74. A suture 78 may be attached to the distal anchor component 70. In one embodiment, a polyethylene terephthalate (PET) suture portion 78 is thermoformed onto locking features in the distal anchor component 70. The distal anchor component 70 may be locally heated to re-flow the suture onto the end of the distal anchor component 70 and into cutouts on the distal anchor component 70. The distal anchor component 70 may be attached to the suture portion 78 through any of several known techniques for bonding a PET material to a nitinol material.

In one embodiment, a mid-section 80 of the distal anchor component 70 provides a structural transition from the head portion 72 to the tail portion 74 and has a portion of a side wall removed in the area of mid-section 80. A further portion of the side wall is removed to define a connector section 82 of the tail portion 74 which extends from the mid-section 80. In one embodiment, this connector section 82 may include a bend that creates the orthogonally oriented configuration. Thus, in its pre-implanted form, the anchor assembly can include a distal anchor component 70 whose initial engagement with a suture portion 78 is generally coaxial.

Still referring to FIG. 4, in one embodiment the proximal anchor component 84 (or “UE”) includes prongs 96 that grip the suture portion 78. The interior structure of the prongs 96 may function to disrupt the surface of the suture portion 78, both pressing into the suture portion 78 and compressing the suture portion 78 therebetween. A tab 98 may be included, extending from one or more of the prongs 96 to help create secure engagement between the proximal anchor component 84 and the suture portion 78. In some examples, the proximal anchor component 84 may comprise stainless steel. In some embodiments, the implant in its entirety may comprise a polymer composition, for example resembling a “T” in whole or in part.

In certain embodiments, the proximal anchor component 84 may be present in the shaft assembly of a delivery device in a configuration that is separate and disconnected from the distal anchor component 70 and the suture portion 78, which may be engaged with each other and contained within the needle assembly. After the distal anchor component 70 and the suture portion 78 have been placed within tissue, the proximal anchor component 84 may be securely engaged with the suture portion 78 to form the fully assembled anchor assembly or implant. To facilitate engagement of the proximal anchor component 84 with the suture portion 78, the proximal anchor component 84 may include, in some examples, a rigid, generally cylindrical back end 95. This rigid, generally cylindrical back end 95 can be used to push the proximal anchor component 84 into engagement with the suture 78 via transfer of the mechanical energy in the handle of the associated delivery device.

The tissue approximation anchors disclosed herein and shown in FIG. 4 may be designed to be useable in a physician's clinical office environment (in contrast to requiring a hospital environment) with a delivery tool. The delivery tool may be used with a 19 F or 20 F sheath in some examples. Additionally, the material selection and construction of the tissue approximation anchor still allows for a subsequent TURP procedure to be performed, if necessary, on the prostate. In this suture-based, tissue approximation technique, a needle delivery mechanism may be used to implant an anchor assembly.

The implants disclosed herein may be delivered to a targeted lobe of a prostate gland using a delivery system that further includes a delivery device comprising a tubular elongate member and at least one hollow delivery needle configured to be advanced therethrough. The needle may have a sharp distal tip configured to pierce the prostate gland, including the outer capsule, along with an inner lumen configured to house the distal anchor component and the suture portion therein, ready for deployment.

Examples of a delivery device may generally include a handle assembly supporting an elongate portion comprising a tubular elongate member in the form of or comprising a shaft assembly. The elongate member may be substantially rigid or flexible and defines a low profile suited to navigate body anatomy, e.g., urethra, to reach an interventional site, e.g., prostatic urethra. An elongate shaft member or assembly may include one or more lumens. Substructure may be provided to maintain a longitudinal profile of the elongate member so that the interventional procedure can progress as intended. Embodiments of the delivery device may also include an endoscope, providing the ability to view the interventional procedure. Using the disclosed systems, insertion of one or more implants in a prostate gland may be performed in an outpatient setting.

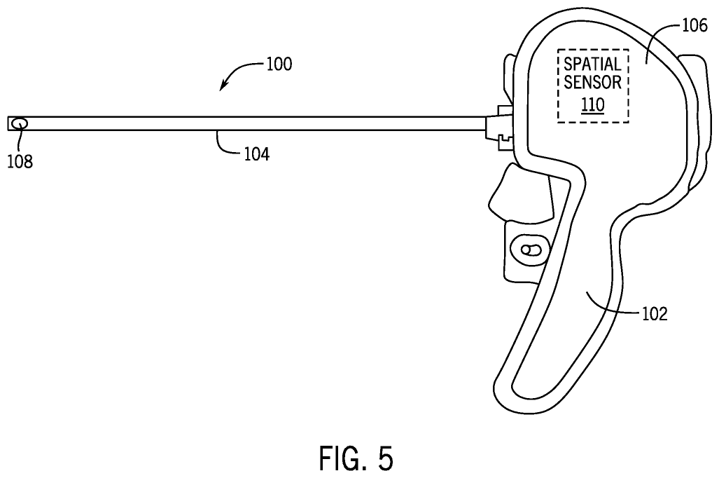

FIG. 5 illustrates one non-limiting example of an implant delivery device 100 having structure configured to gain access to an interventional site and deploy one or more prostatic implants, such as implant 10. As shown, the implant delivery device 100 may include a handle assembly 102 connected to a tubular elongate member 104 (or shaft assembly), which may surround an elongate delivery needle defining an inner lumen and arranged coaxially to the elongate member 104.

The implant delivery device 100 may further include a number of subassemblies configured to deliver and employ one or more implants at a target site. A handle case assembly 106 is also shown, including handle parts that form part of the handle assembly 102. The handle assembly 102 is sized and shaped to fit comfortably within an operator's hand and can be formed from conventional materials. Windows can be formed in the handle case assembly 106 to provide access to internal mechanisms of the device so that a manual override is available to the operator in the event the interventional procedure needs to be abandoned.

In some examples, the elongate member 104 (or shaft assembly) may define at least one inner lumen sized and configured to accommodate longitudinal insertion of at least the hollow delivery needle and prostatic implant therethrough, with the distal anchor member or CT of the implant nestled within the delivery needle, and the proximal anchor component or UE included in the elongate member 104 in a delivery configuration. In some examples, the CT and middle portion or suture may be enclosed within a first lumen of the elongate member 104, and the UE may be enclosed within a second lumen of the elongate member 104. The elongate member 104 may have a shape and/or flexibility configuring it to navigate through a urethra without kinking or puncturing the urethral wall. In some examples, the elongate member 104 may be substantially rigid, such that it maintains an approximately straight configuration during its insertion through the urethra. According to such examples, the distal portion of the elongate member 104 may be angled toward or away from various anatomical features surrounding the urethra, e.g., one or more lobes of the prostate gland, by adjusting the angular orientation of the proximal end of the elongate member 104 outside the body. The distal end of the elongate member 104 may comprise smooth, blunt, and/or beveled surfaces to avoid puncturing the urethral wall. Embodiments may include a sidewall opening 108 (or exit port) defined by a distal portion of the elongate member 104. The sidewall opening 108 may be sized and configured to accommodate passage of the hollow delivery needle therethrough.

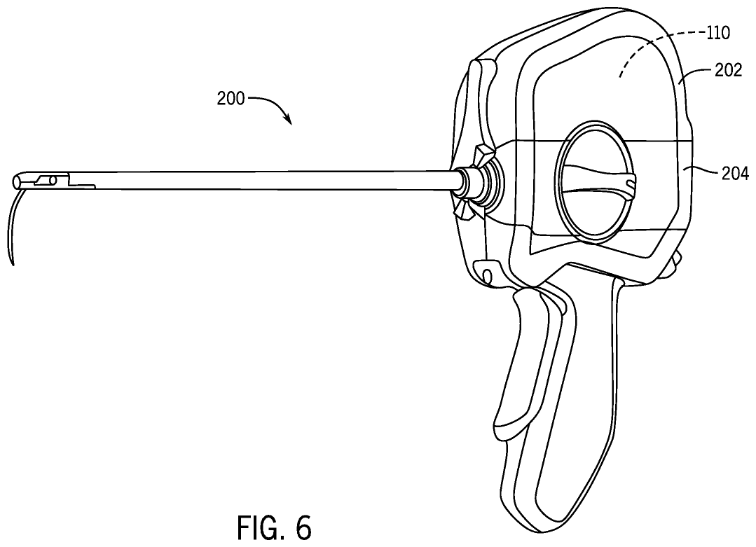

In some examples, a delivery device may include a handle assembly and a removable cartridge 204 configured to couple therewith. For instance, FIG. 6 illustrates a non-limiting example of a delivery device 200 that includes a handle assembly 202 and complementary cartridge 204 that can be used to deliver one or more implants to prostatic tissue, for example pursuant to a method of treating benign prostatic hyperplasia. The handle assembly 202 includes sources of mechanical energy that may be transferred to the cartridge 204 to deploy the anchor assembly within the cartridge 204. The handle assembly may be configured such that the energy can be restored to these mechanical energy sources while the first cartridge 204 is being used and/or prior to the insertion of a second cartridge 204. The handle assembly 202 may be designed to reliably deliver energy to multiple cartridges 204 in sequence before a new handle assembly is required.

Embodiments of the delivery device may include a variety of additional or substitute components and subassemblies, such as those described in U.S. Pat. Nos. 10,130,353, 11,298,115, and U.S. Patent Application Publication No. 2021/0378658, the entire contents of each of which are incorporated by reference herein.

Each implant or component thereof may be provided in a separate delivery device or cartridge 204 configured to couple therewith. Where a cartridge 204 containing an implant 10 is used, the cartridge 204 may be loaded into a delivery device, which may then be activated by a user to deploy the implant into the targeted tissue by transferring mechanical energy to the cartridge 204 or internal subassembly. The delivery device, or at least its elongate shaft assembly, may then be removed from the patient, and the spent cartridge 204 replaced with a new cartridge 204 containing a second implant. The delivery device (or more specifically, the shaft assembly) may then be re-inserted and the deployment process repeated. Multiple implants are often necessary to complete a single procedure, thus necessitating multiple cartridge 204 exchanges and the associated removals/reinsertions of the shaft assembly. In some embodiments, a delivery device may be configured to simultaneously hold and serially deploy multiple implants or be reloaded with multiple implants during a procedure, for example via cartridge exchange, in a manner that does not require the shaft assembly to be removed between deployments. Embodiments of the delivery devices disclosed herein may thus be configured to deploy multiple implants without removing the elongate shaft assembly from the urethra of a patient. According to such embodiments, which may be considered multi-fire embodiments, the delivery devices include various subassemblies mobilized via one or more actuators or manually accessible structures, the operation of which is coordinated and synchronized to ensure accurate and precise implantation of each implant. Non-limiting examples of such multi-fire devices and device components are described in U.S. Patent Application Publication No. 2025/0339134, the entire contents of which are incorporated by reference herein.

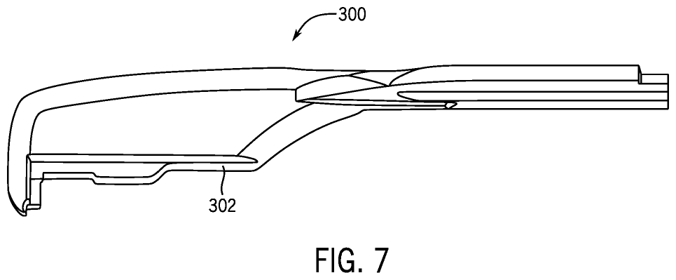

FIG. 7 provides a side view of a portion of a distal tip of a portion of an elongate shaft assembly, which may constitute or be coupled with a handle assembly or a cartridge 204 in various embodiments. As shown, the shaft distal portion 300 may include a shaft distal portion exit port 302 from which a distal portion of a delivery needle (not shown) emerges when the needle distal portion is extended from the shaft distal portion 300. The exit port 302 may therefore constitute the distal terminus of an elongate shaft lumen, which may define an effective radius of curvature as the lumen transitions from running along the long axis of the shaft assembly to running in a direction transverse to the long axis of the shaft assembly in some examples. Additional, non-limiting features of an example of a shaft assembly or distal portion thereof are described in U.S. Pat. No. 11,298,115.

At or near the exit port 302, a UE may be attached to each of one or more sutures implanted and tensioned through a targeted lobe of a prostate gland. After or during the UE attachment process, the excess suture proximal to the attached UE may be cut. Visualizing one or more steps involved in the implant deployment process, including navigation of the shaft assembly to a treatment site, positioning of the distal end of the shaft assembly adjacent to a targeted lobe, insertion of the distal tip of a delivery needle containing one or more implant components into the targeted tissue, tightening of the suture, and/or placement of the UE, among other steps, may be imperative to completing the treatment procedure effectively.

Embodiments of one or more implant delivery systems disclosed herein may include one or more visualization components, assemblies, or systems configured to enable real-time viewing of a procedure at or near a treatment site, for instance within or near a prostatic urethra. In some examples, one or more visualization components can be included, integrated, or otherwise coupled with one or more components of the implant delivery device, such as within at least a portion of the elongate shaft assembly, where the component(s) can provide a real-time view of the procedure from within the patient. Examples may provide a view of the needle exiting the device and advancing into the targeted prostatic lobe. Examples may also be configured to provide a real-time view of the suture tensioning and/or UE attachment process. Non-limiting examples of various visualization components, assemblies, and systems are described in U.S. Patent Application Publication No. 20250339139, the entire contents of which are incorporated by reference herein.

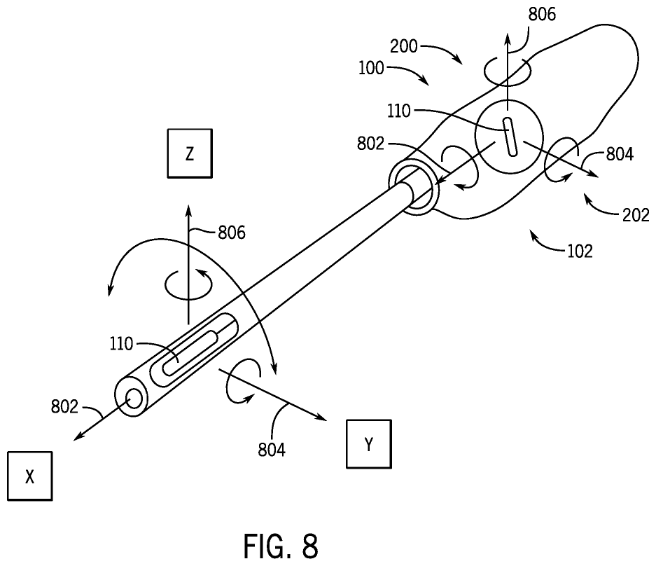

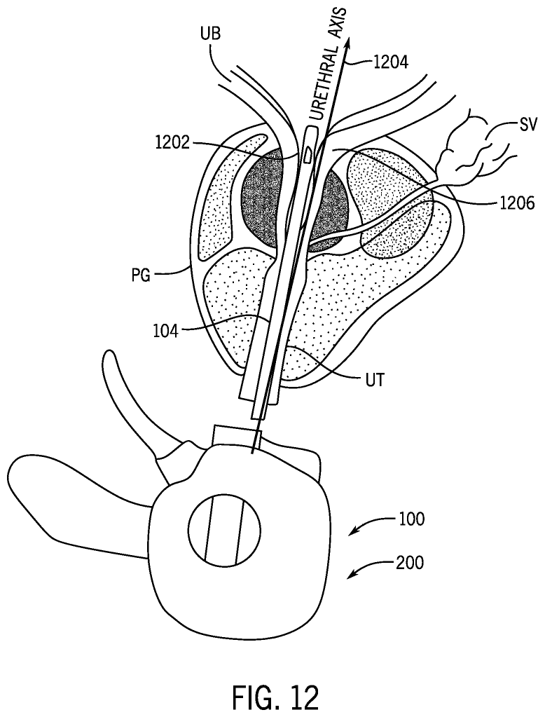

Turning to FIG. 8, but with reference to FIG. 5 and FIG. 6, either or both of the implant delivery device 100 and/or implant delivery device 200 may include, or be coupled to a spatial sensor 110. The spatial sensor 110 is any device that can determine a location and/or orientation of at least a portion of the implant delivery device 100 and/or implant delivery device 200 with respect to a reference 1206 (see e.g., FIG. 12). In many embodiments, the reference 1206 may be an anatomical feature of the patient 1, such as a bladder neck 1202, etc. In some embodiments, the reference 1206 may be a fiducial marker, or other non-anatomical reference feature, point, area, or region.

As shown for example in FIG. 5 and FIG. 6, the spatial sensor 110 may be coupled to the handle assembly 102 and/or handle assembly 202, respectively. The spatial sensor 110 may be a low profile device adhered to the outer surface of the handle assemblies, or may be enclosed in the handle case assembly 106 (e.g., as shown for example in FIG. 8). As shown for example in FIG. 8, the spatial sensor 110 may also be enclosed in or coupled to the elongate member 104 of either or both of the implant delivery device 100 or implant delivery device 200.

In some embodiments, the elongate member 104 may define a channel or lumen configured to receive a cystoscope or other endoscopic instrument that extends along the length of the elongate member 104 for visualization of the operating field during a procedure. The cystoscope may be removable from the elongate member 104 and may be transferable between different handle assemblies or cartridges during a procedure, for example when deploying multiple implants using a single-shot delivery device or a cartridge-based system. In some embodiments, the spatial sensor 110 may be coupled to or enclosed within the cystoscope or endoscopic instrument that is inserted into the channel of the elongate member 104, rather than being coupled directly to the elongate member 104 itself. According to such embodiments, the spatial sensor 110 may move with the cystoscope when the cystoscope is transferred between handle assemblies or cartridges, allowing the same spatial sensor 110 to provide tracking data across multiple implant deployments without requiring a separate spatial sensor 110 for each handle assembly or cartridge. In some embodiments, the cystoscope or endoscopic instrument may remain within the handle assembly throughout the entire procedure, as all implants may be contained within the device, thereby eliminating the need for repeated insertion and removal of the cystoscope.

As described further with respect to the system 900, in some embodiments, the spatial sensor 110 may be an inertial measurement unit or “IMU”. An IMU is a sensor device used to measure and report on a specific force, angular rate, and often the magnetic field surrounding the implant delivery device 100 or implant delivery device 200, including real-time tracking of such data. An IMU can include accelerometers, gyroscopes, and/or magnetometers and/or combinations thereof to provide comprehensive data on movement, position, location, and/or orientation of the implant delivery device 100 or implant delivery device 200.

As described further with respect to the system 1000, in some embodiments, the spatial sensor 110 may be an electromagnetic tracking or “EM” device. An EM device may be used in conjunction with a field generator 1002 to provide real-time tracking of an implant delivery device 100 and/or implant delivery device 200. The system 1000 typically includes a field generator 1002 that emits a controlled electromagnetic field 1004 and one or more spatial sensors 110 affixed to, or included in, the implant delivery device 100 and/or implant delivery device 200. The electromagnetic field 1004 is designed to cover the surgical area within a field range 1006, allowing for the precise monitoring of instrument positions and orientations relative to the patient's 1 anatomy.

In some embodiments, the spatial sensor 110 is a 6-axis device (either/both an IMU or EM) that measures data relative to orthogonal axes aligned to a three-dimensional Cartesian coordinate system: typically denoted as the x-axis 802, y-axis 804, and z-axis 806. In many embodiments, when the spatial sensor 110 includes an IMU, each of these orthogonal axes is equipped with an accelerometer and a gyroscope. The accelerometers measure linear acceleration along the x-axis 802, y-axis 804, and z-axis 806, which may correspond to forward/backward, left/right, and up/down movements, respectively. The gyroscopes measure rotational rates about the x-axis 802, y-axis 804, and z-axis 806, capturing roll, pitch, and yaw motions. Collectively, these measurements provide six degrees of freedom (DOF), enabling the spatial sensor 110 to comprehensively capture the translational and rotational dynamics of all or a portion of the implant delivery device 100 and/or implant delivery device 200 in three-dimensional space.

When the spatial sensor 110 is an EM device, the spatial sensor 110 performs similar measurements as an IMU by exploiting the principles of electromagnetic induction. The field generator 1002 creates an electromagnetic field 1004 of known properties within the field range 1006 in the surgical area. Each spatial sensor 110 attached to the implant delivery device 100 and/or implant delivery device 200 is equipped to detect the disturbances within the electromagnetic field 1004 as the implant delivery device 100 and/or implant delivery device 200 moves through the electromagnetic field 1004. By analyzing these disturbances, the system 1000 can determine the precise location and orientation of the sensor 110 relative to the field generator 1002. This information is continuously updated and fed into the system 1000, which processes the data to provide a dynamic and accurate depiction of a position, location, and/or orientation of surgical instruments in real-time.

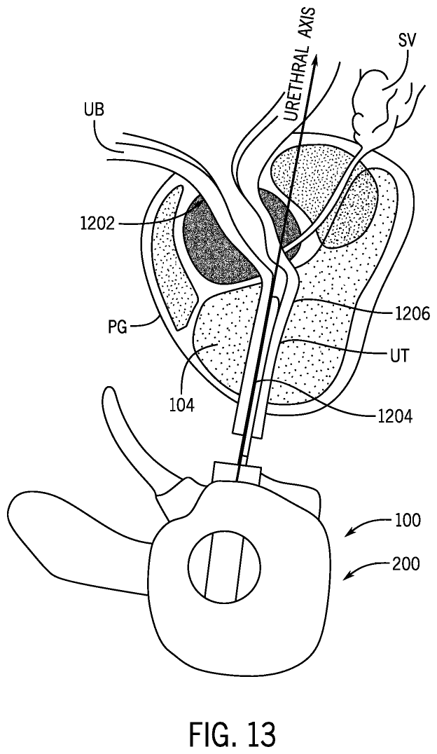

The spatial sensor 110 tracks the motion of the implant delivery device 100 and/or implant delivery device 200 by measuring linear and/or rotational acceleration and/or rotational velocity (in the vector sense, i.e., a combination of speed and direction). For example, the accelerometers in an IMU detect changes in velocity, while the gyroscopes measure the rate of rotation around an axis (e.g., a urethral axis 1204 described with respect to FIG. 12 and FIG. 13), providing a system (such as the system 900 and system 1000 described herein) with motion data.

In some embodiments, tracking of the position, location, orientation, velocity, and/or acceleration of the implant delivery device 100 and/or implant delivery device 200 may be performed using image-based techniques applied to video feed captured by the image sensor 912 during cystoscopy. For example, Simultaneous Localization and Mapping (SLAM) algorithms may be employed to build a map of the urethral and prostatic environment while simultaneously tracking the position and orientation of the elongate member 104 within that environment. In some examples, visual odometry techniques may be used to estimate device motion by analyzing changes between successive video frames captured by the image sensor 912. These image-based tracking techniques may be used as an alternative to the spatial sensor 110, or may be used to supplement and fuse with data from the spatial sensor 110 to provide improved tracking accuracy and robustness. The processing element 2302 may execute algorithms to process the video frames and derive position, orientation, velocity, and/or acceleration data, which may then be integrated with or used in place of data from the spatial sensor 110 for intra-operative guidance 1106.

The motion data can then be processed, e.g., by a processing element 2302, to derive the current acceleration, velocity, position, and/or orientation of the implant delivery device 100 and/or implant delivery device 200. In some embodiments, magnetometers are included in the IMU to offer data on the heading of the implant delivery device 100 and/or implant delivery device 200 relative to the magnetic field of the Earth, enhancing the accuracy of the orientation measurements.

The process of locating at least a portion of the implant delivery device 100 and/or implant delivery device 200 using an IMU involves integrating the data collected from its sensors. By continuously measuring acceleration and rotational rates, the IMU calculates the change in position and orientation over time. This data integration allows the IMU to track the implant delivery device 100 and/or implant delivery device 200 trajectory, attitude, position, location, and/or orientation. However, due to sensor noise and drift, in the integration process, the position and orientation estimates can accumulate error over time. To mitigate such errors, the systems 900 and system 1000 may reset, calibrate, or “zero” the IMU based on the reference 1206. Similarly, when the spatial sensor 110 is an EM device, the location, position, and/or orientation of the spatial sensor 110 may be zeroed, calibrated, or reset based on the reference 1206. In some embodiments, the clinician may manually identify the reference 1206, place a portion of the implant delivery device 100 and/or implant delivery device 200 adjacent to the reference 1206 and activate a user input (e.g., a button or touch screen region, etc.) to reset the location, position, and/or orientation of the spatial sensor 110.

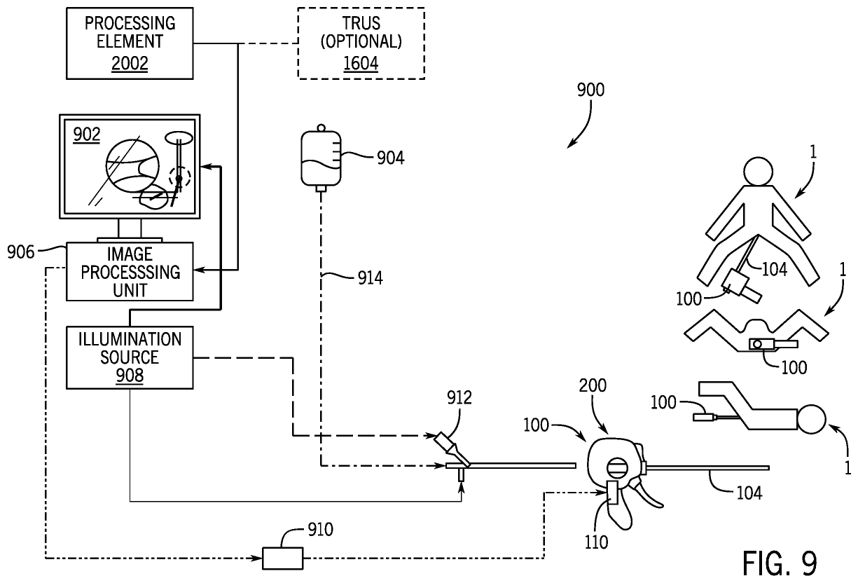

Turning to FIG. 9, a system 900 is shown. The system 900 is suitable for use with any implant delivery device disclosed herein, including the implant delivery device 100 or the implant delivery device 200, and including implant devices configured to deploy multiple implants without removing the associated shaft assemblies from a patient. As described herein, embodiments of the implant delivery device, e.g., the implant delivery device 100 and/or implant delivery device 200, may include a spatial sensor 110. The system 900 may be specifically adapted for use with an IMU-type spatial sensor 110.

The system 900 includes an implant delivery device 100 and/or an implant delivery device 200, a display 902, an image processing unit 906, an illumination source 908, an optional sensor interface unit 910, and an image sensor 912. An IV bag 904 may be included with the system 900 to provide a procedural liquid such as a saline solution to the implant delivery device 100 and/or implant delivery device 200 to irrigate a procedural site, via a conduit 914. An introducer sheath positioned within a urethra and coupled with the delivery device during a procedure may provide a lumen for injecting the irrigant through the urethra to a treatment site. The treatment site may include a prostatic urethra, which may be constricted or impinged due to enlargement of the surrounding prostate tissue.

FIG. 9 shows various views of the body of a patient 1 in a procedural position, with respect to an implant delivery device 100 or implant delivery device 200 (although only an implant delivery device 100 is shown in FIG. 9, the implant delivery device 200 is equally applicable.)

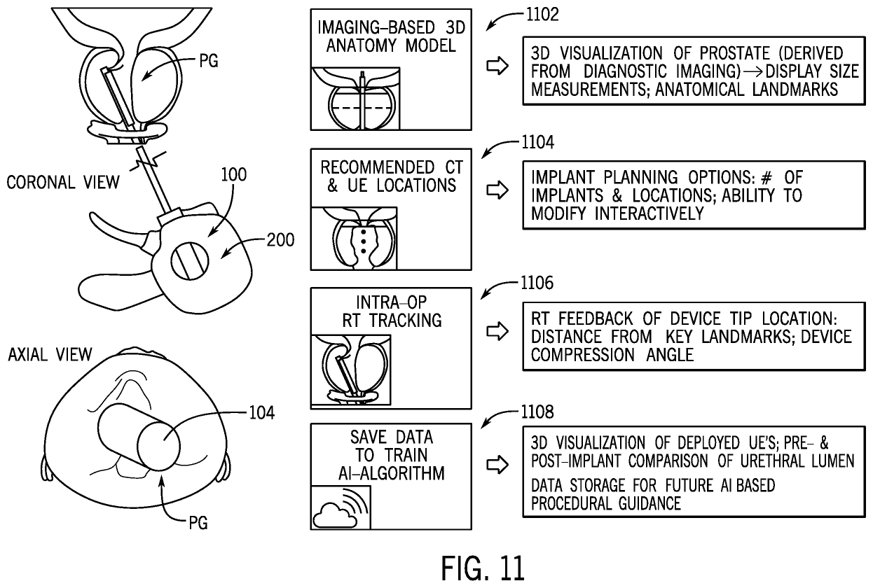

During a prostate procedure, e.g., implant deployment, the system 900 may provide light to the procedural site via the illumination source 908. The illumination source 908 may be any suitable source of light, such as a light emitting diode, laser, etc. The image sensor 912, which may include any suitable device such as a still and/or video camera suitable to capture images of the procedural site, may relay those images to an image processing unit 906 for presentation on the display 902. A processing element 2302 of the system 900 may also analyze the images captured by the camera to provide or update intra-operative guidance 1106 and/or pre-operative planning guidance 1104 (described with respect to FIG. 11-FIG. 16).

The sensor interface unit 910 may communicate with the spatial sensor 110 to determine a position, location, and/or orientation of at least a portion of an implant delivery device, e.g., the implant delivery device 100 and/or implant delivery device 200, as disclosed herein.

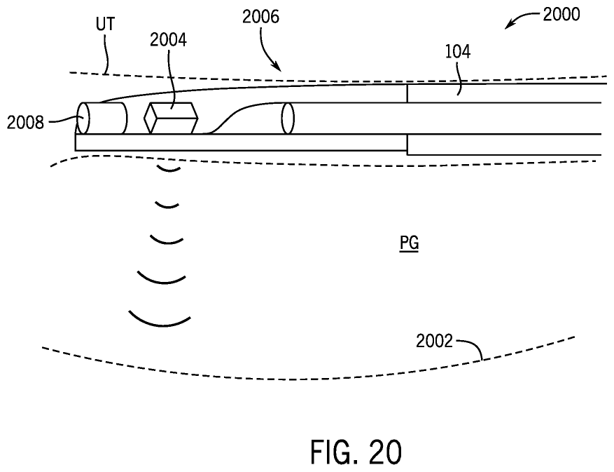

With attention to FIGS. 20-22, the system 900 may optionally include a trans-rectal ultrasound (TRUS 1604) to provide live intraoperative imaging in addition to the images captured by the image sensor 912. In some embodiments, the system 900 may include a trans-urethral ultrasound (TUUS) co-located on the shaft of the implant delivery device 100 and/or implant delivery device 200, such as the sensor 2004 of the system 2000 described with respect to FIGS. 20-22. In some embodiments, other ultrasound modalities or non-ultrasound imaging modalities may also be used to provide live intraoperative imaging to supplement the images captured by the image sensor 912.

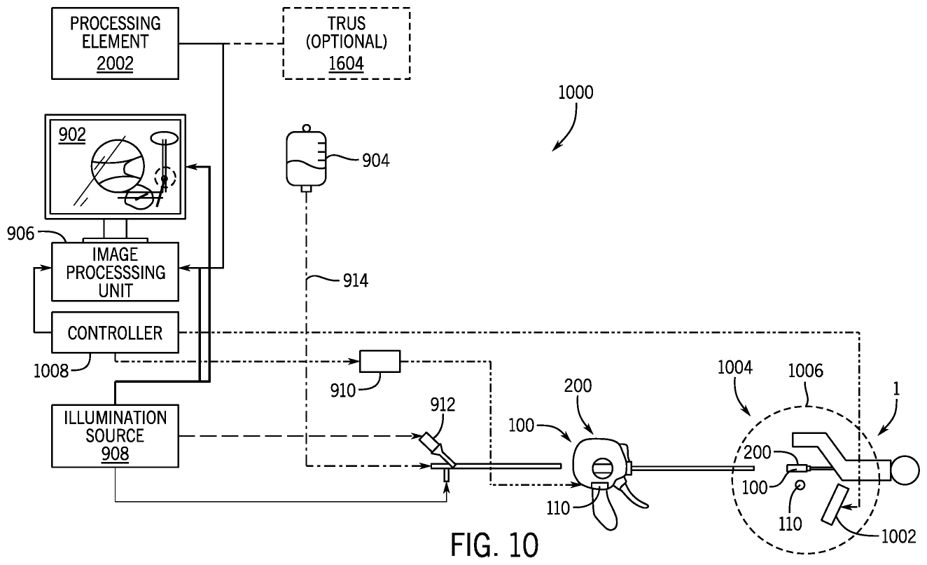

Turning to FIG. 10, a system 1000 is disclosed. The system 1000 is similar to the system 900 in many aspects. For example, the system 1000 may include an implant delivery device, e.g., implant delivery device 100 and/or implant delivery device 200, a spatial sensor 110, a display 902, an IV bag 904, an image processing unit 906, an illumination source 908, an optional sensor interface unit 910, an image sensor 912, and a conduit 914, as described with respect to the system 900.

Similarly, the system 1000 may display and/or analyze images of the anatomy of patient 1 as described with respect to the system 900.

Where the system 1000 may differ from the system 900 is that the system 1000 may be particularly adapted for use with an EM-type spatial sensor 110.

The system 1000 includes a field generator 1002 that creates an electromagnetic field 1004 with a field range 1006, a tracking control unit 1008, and a processing element 2302. The tracking control unit 1008 communicates with the optional sensor interface unit 910 and the field generator 1002 to determine the location, position, and/or orientation of at least a portion of an implant delivery device, e.g., the implant delivery device 100 and/or implant delivery device 200, with respect to the body of the patient 1.

With attention to FIGS. 20-22, the system 1000 may optionally include a trans-rectal ultrasound (TRUS 1604) to provide live intraoperative imaging in addition to the images captured by the image sensor 912. In some embodiments, the system 1000 may include a trans-urethral ultrasound (TUUS) co-located on the shaft of the implant delivery device 100 and/or implant delivery device 200, such as the sensor 2004 of the system 2000 described with respect to FIGS. 20-22. In some embodiments, other ultrasound modalities or non-ultrasound imaging modalities may also be used to provide live intraoperative imaging to supplement the images captured by the image sensor 912.

Turning to FIG. 11, examples of outputs of either or both of the system 900 and/or system 1000 are described. The system 900 and/or system 1000 may generate an anatomical model 1102 of the anatomy of patient 1, e.g., including a prostatic urethra and surrounding tissues. For example, a processing element 2302 of the systems may generate a 3D anatomical model 1102 of the body of patient 1 or portion thereof by processing volumetric data acquired from imaging modalities like CT, MRI, and/or ultrasound (particularly transrectal ultrasound or “TRUS” in the case of prostate procedures). Such imaging techniques produce high-resolution cross-sectional images that processing element 2302 integrates and assembles into a three-dimensional representation. In some embodiments, program instructions stored on a memory component 2308 may be executed by processing element 2302 that process image data by interpreting grayscale values or signals that correspond to different tissue densities or properties, thereby differentiating between various anatomical structures. The program instructions may then perform image segmentation to isolate specific structures or regions of interest, such as organs or pathological areas, facilitating the creation of a detailed, interactive 3D anatomical model 1102. The anatomical model 1102 can be used for diagnostic purposes, pre-surgical planning, intra-operative guidance 1106, etc., thereby improving the placement of implants 10 and overall procedural outcome for the patient 1 compared to prior methods and systems of treating BPH.

In some embodiments, the processing element 2302 may fuse the anatomical model 1102 generated from volumetric data with cystoscopy video captured by the image sensor 912 to generate a combined model that includes both an internal model of the urethral channel and an exterior model of the prostatic capsule. The internal model may be derived from real-time or recorded cystoscopy video frames, while the exterior model may be derived from volumetric imaging data such as CT, MRI, or TRUS. By combining these data sources, the system 900 and/or system 1000 may provide a more comprehensive representation of the anatomy of patient 1 for treatment planning and intra-operative guidance 1106.

In some embodiments, the combined model may be tagged or annotated using computer vision techniques to recognize anatomical landmarks and features that may impact treatment planning. For example, the processing element 2302 may execute algorithms to identify features such as an obstructive median lobe, a high bladder neck, or intravesicular protrusion within the cystoscopy view 1404 or the anatomical model 1102. Recognition of such features may assist the clinician in determining optimal implant 10 placement locations and adjusting the planning guidance 1104 accordingly.

In some embodiments, when volumetric imaging data is not available, the processing element 2302 may generate an internal model of the urethral channel solely from cystoscopy video captured by the image sensor 912. In such cases, an exterior model of the prostatic capsule may be imputed from anatomical templates stored in the memory component 2308. Simple scaling techniques may be applied to the templates based on measurements derived from the cystoscopy video or other available patient data to approximate the anatomy of patient 1. This approach may enable the system 900 and/or system 1000 to provide planning guidance 1104 and intra-operative guidance 1106 even in the absence of preoperative volumetric imaging.

In some embodiments, the system 900 and/or system 1000 may communicate different levels of fidelity or confidence to the user based on the data inputs used to generate the anatomical model 1102. For example, regions of the model derived from high-resolution volumetric data may be displayed with a first visual indicator, while regions derived from cystoscopy video or imputed from templates may be displayed with a second visual indicator. The visual indicators may include different color overlays, line widths, transparency levels, or other graphical representations that convey the relative confidence or accuracy of different portions of the anatomical model 1102. Such visual differentiation may assist the clinician in interpreting the planning guidance 1104 and intra-operative guidance 1106 and in making informed decisions during the procedure.

The system 900 and/or system 1000 may generate, via the processing element 2302, a planning guidance 1104 that shows a clinician how many implants 10 to place in the prostate and where to place them, for an improved surgical outcome compared to prior methods and systems.

The system 900 and/or system 1000 may generate, via the processing element 2302, intra-operative guidance 1106 that a clinician can use during a BPH treatment procedure to adjust for movements or changes in the anatomy of patient 1, differences discovered between the anatomical model 1102 and the actual anatomy, failure of an implant 10 to be properly placed, etc. See, e.g., FIG. 13 and related description. Thus, the systems disclosed herein provide for improved patient 1 outcomes compared to prior methods and systems of treating BPH.

The system 900 and/or system 1000 may generate, via the processing element 2302, training data 1108 used to train an artificial intelligence “AI” algorithm to provide continual improvement of the anatomical model 1102, the planning guidance 1104, and/or the intra-operative guidance 1106. For example, program instructions stored on memory component 2308 may implement an AI algorithm or machine learning model, to analyze medical images captured by the image sensor 912, identify and segment anatomical structures of the body of patient 1. In some embodiments, convolutional neural networks (CNNs) may be implemented by the system 900 and/or system 1000 and may interpret complex imaging data from MRI, CT, and/or TRUS scans, aiding clinicians in planning by providing detailed anatomical models 1102. In some embodiments, reinforcement learning models may be implemented by the system 900 and/or system 1000 to simulate surgical procedures, offer predictive insights into alternative surgical strategies and outcomes. During a procedure, the AI models can support decision-making and navigation of the implant delivery device 100 and/or implant delivery device 200 by providing updated (often in real-time) planning guidance 1104.

Turning to FIG. 12, a schematic representation of the prostate of a patient 1 is shown including an inserted implant delivery device 100 and/or implant delivery device 200. An elongate member 104, e.g., shaft assembly, of the implant delivery device is inserted into the urethra, along a urethral axis 1204. In the example shown, the tip of the elongate member 104 is inserted up to the bladder neck 1202. As described with respect to the spatial sensor 110, a clinician may use the bladder neck 1202 as a reference 1206 to “zero”, reset, or calibrate the location, position, and/or orientation of the implant delivery device 100 and/or implant delivery device 200. For example, the clinician may insert the tip of the elongate member 104 up to, or into, or slightly past the bladder neck 1202. The clinician may also rotate the implant delivery device 100 and/or implant delivery device 200 to a rotational position with respect to the prostate, such as a 12 o'clock, 3 o'clock, 6 o'clock, 9 o'clock position, etc. Once the implant delivery device 100 and/or implant delivery device 200 is in the desired translational and/or rotational position, the clinician, or another person, may activate a user input of the system 900 and/or system 1000 to cause the respective system to “zero”, reset, or calibrate the location, position, and/or orientation of the implant delivery device 100 and/or implant delivery device 200.

Additionally, or alternately, placing the implant delivery device 100 and/or 200 as shown may orient the implant delivery device 100 and/or implant delivery device 200 to the anatomical model 1102, such as to provide for the system to provide and track the progress of the BPH procedure via intra-operative guidance 1106.

In some embodiments, particularly when an AI algorithm is used with the system 900 and/or system 1000, the systems may automatically recognize the bladder neck 1202 or other reference 1206 based on one or more images captured by the image sensor 912 and may automatically “zero”, reset, or calibrate the location, position, and/or orientation of the implant delivery device 100 and/or implant delivery device 200 or orient these to the anatomical model 1102.

Turning to FIG. 13, an example of the system 900 and/or system 1000 tracking changes in the anatomy of patient 1 during a BPH procedure is shown. For example, a change in the urethral axis 1204 compared to the anatomical model 1102 may have occurred (either due to anatomical changes and/or inaccuracies in the anatomical model 1102 and/or the images used to create the model). The systems may track such changes or other changes based on the spatial sensor 110 as described herein.

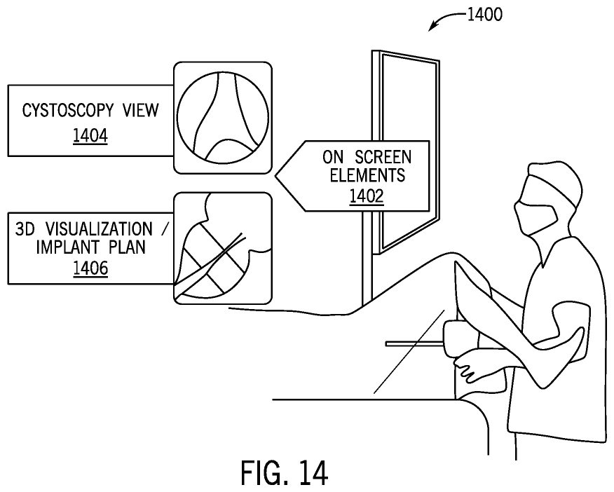

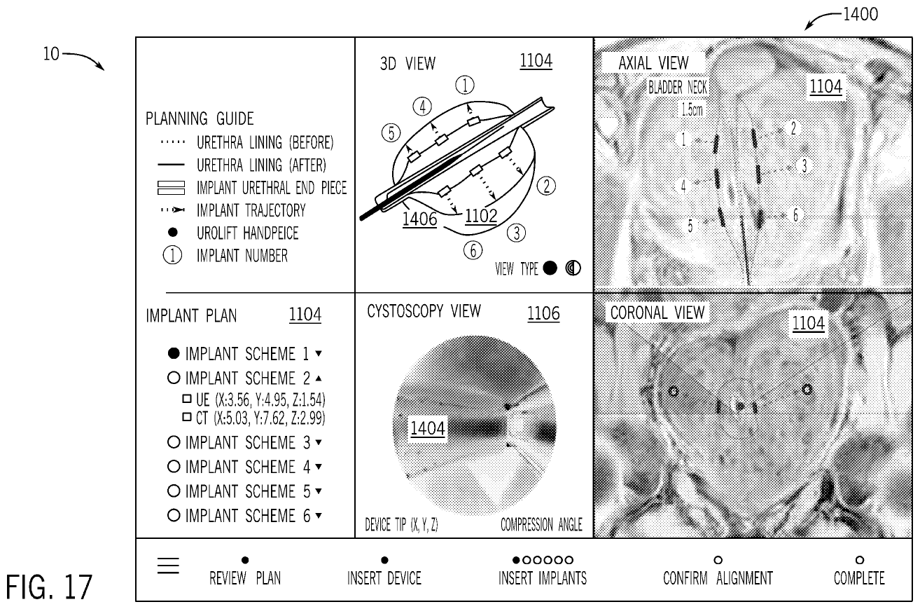

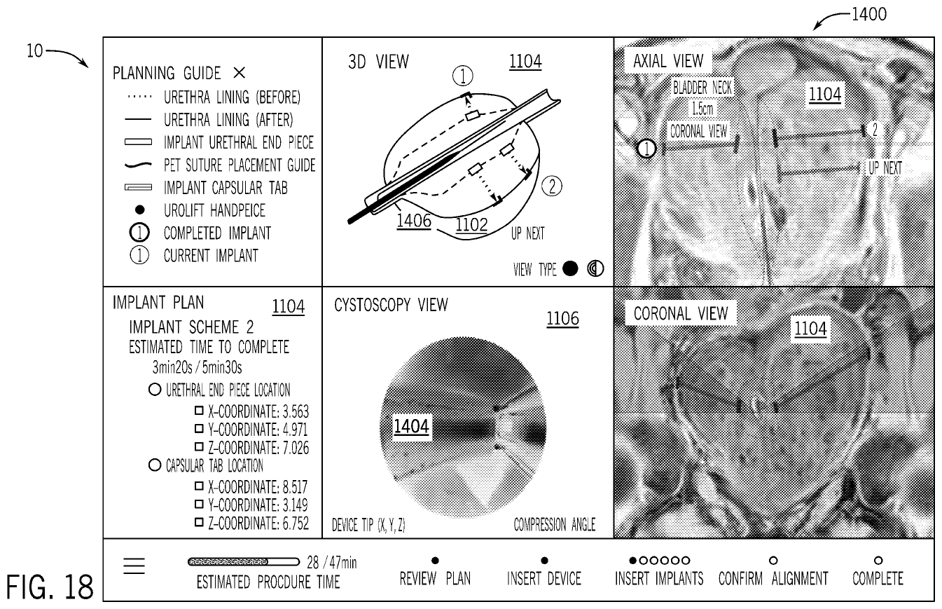

FIG. 14 shows an example of on screen elements 1402 that may be presented by the system 900 and/or system 1000, via a user interface 1400, e.g., on a display 902. As shown for example in FIG. 14 the systems may show a cystoscopy view 1404 and/or a 3D visualization/implant plan 1406. The 3D visualization/implant plan 1406 may combine a view of the anatomical model 1102 with the proposed locations of the implants 10 (see also, FIG. 17 and FIG. 18).

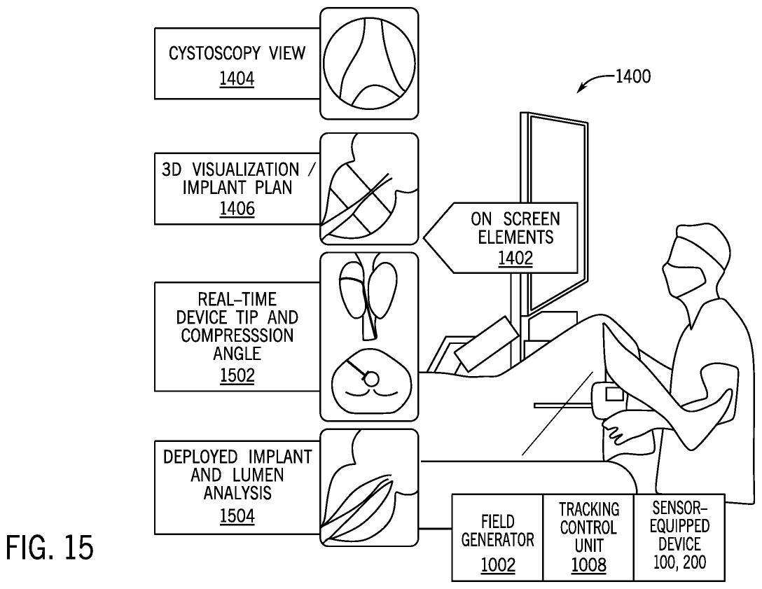

FIG. 15 is similar to FIG. 14 but the on screen elements 1402 of the user interface 1400 additionally include real-time device tip and compression angle 1502 (e.g., as provided by a spatial sensor 110), and/or a deployed implant and lumen analysis 1504. FIG. 15 may be particularly suited to a system 1000 including an EM-type spatial sensor 110, as the example shown includes field generator 1002 and a tracking control unit 1008, an implant delivery device 100 and/or implant delivery device 200 with a spatial sensor 110.

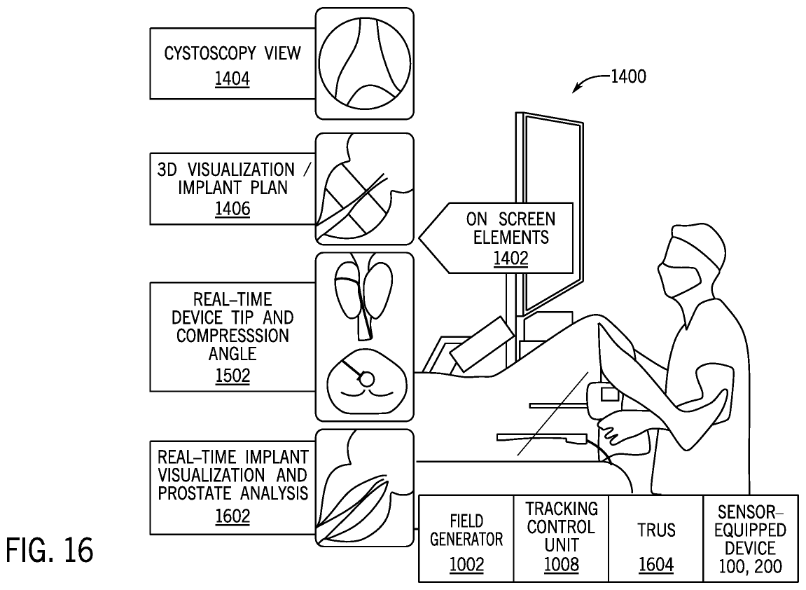

FIG. 16 is similar to FIG. 13 and FIG. 14 but the on screen elements 1402 of the user interface 1400 additionally include a real-time implant visualization and prostate analysis 1602. For example, the embodiment of the system shown in FIG. 16 includes a trans-rectal ultrasound to provide live intra-operative guidance 1106 and imaging of the prostate.

Turning to FIG. 17 and FIG. 18, examples of planning guidance 1104 and intra-operative guidance 1106 are shown via a user interface 1400. The planning guidance 1104 may include an image of the anatomical model 1102 overlaid with proposed locations for implant 10 (e.g., numbered 1-6 in FIG. 17) placement. The user interface 1400 may distinguish between two use cases: planned implant locations that are suggested by the treatment plan but not yet placed, and implants that have already been placed. For example, planned implant locations may be displayed with a first visual indicator (e.g., a dashed line, outline, or first color), while placed implants may be displayed with a second visual indicator (e.g., a solid line, filled marker, or second color) to clearly differentiate between the two states. The user interface 1400 may include a mechanism for switching a planned implant to a placed implant status. For example, the system 900 and/or system 1000 may detect when an implant has been fired based on sensor data, actuation signals from the implant delivery device 100 and/or implant delivery device 200, or user input, and automatically update the display to reflect the placed status of the implant. Additionally, the user interface 1400 may provide functionality for handling implants that are not placed according to plan. For example, if an implant is deployed at a location or orientation that differs from the planned location or orientation, the system 900 and/or system 1000 may detect the deviation and provide options for adjusting the treatment plan accordingly, such as recalculating remaining implant positions, suggesting corrective implant placements, or allowing the user to manually modify the plan. A view of the anatomical model 1102 may be combined with the proposed locations of the implants 10 and/or the placed implants in a unified 3D visualization/implant plan 1406. The planning guidance 1104 may also show images captured by a CT, MRI, or TRUS used in planning the procedure and generating the anatomical model 1102. For example, the planning guidance 1104 may include an axial, coronal, or other view of the anatomy of patient 1. The images may also be overlaid with planned implant 10 locations and/or placed implant 10 locations. The intra-operative guidance 1106 may show a cystoscopy view of the anatomy of patient 1, intra-operatively. The system 900 and/or system 1000 may update the planning guidance 1104 and/or intra-operative guidance 1106 during the procedure, including updating the status of implants from planned to placed as each implant is deployed. The intra-operative guidance 1106 and/or planning guidance 1104 may show the positions and/or conditions of the urethra both before the procedure and an actual or projected position or condition of the urethra after the procedure.

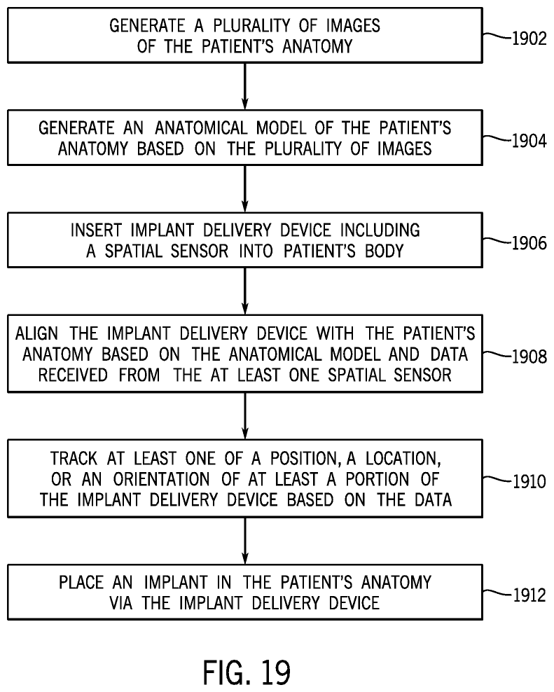

FIG. 19 illustrates a flowchart of a method 1900 of treating a prostate gland using the systems and devices disclosed herein, consistent with the system architectures shown in FIG. 9 and FIG. 10 and the implant delivery devices shown in FIG. 5 and FIG. 6. The method 1900 may be performed using the system 900, system 1000, and/or system 2000 in conjunction with the implant delivery device 100 and/or implant delivery device 200.

At operation 1902, a plurality of images of the anatomy of a patient 1 are generated. For example, the plurality of images may include MRI images, CT images, and/or transrectal ultrasound (TRUS) images of the prostate gland PG and surrounding anatomical structures, such as the urinary bladder UB, urethra UT, and prostatic capsule PC as shown in FIGS. 1A and 3G. In some embodiments, the images may be captured using the TRUS 1604 shown in FIG. 9 and FIG. 10. The images may include cross-sectional views in coronal, axial, and/or sagittal planes to provide comprehensive volumetric data of the patient's anatomy. At operation 1902, a plurality of images of the anatomy of a patient 1 are generated. For example, the plurality of images may include MRI images, CT images, and/or transrectal ultrasound (TRUS) images of the prostate gland PG and surrounding anatomical structures, such as the urinary bladder UB, urethra UT, and prostatic capsule PC as shown in FIGS. 1A and 3G. In some embodiments, the images may be captured using the TRUS 1604 shown in FIG. 9 and FIG. 10. In some embodiments, the images may be captured using a trans-urethral ultrasound (TUUS) co-located on the shaft of the implant delivery device 100 and/or implant delivery device 200, such as the sensor 2004 of the system 2000 described with respect to FIGS. 20-22. The images may include cross-sectional views in coronal, axial, and/or sagittal planes to provide comprehensive volumetric data of the patient's anatomy.

At operation 1904, an anatomical model 1102 of the anatomy of the patient 1 is generated based on the plurality of images. As described with respect to FIG. 11, the processing element 2302 may generate a 3D anatomical model 1102 by processing volumetric data acquired from the imaging modalities. For example, the processing element 2302 may execute program instructions stored on the memory component 2308 that perform image segmentation to isolate specific structures or regions of interest, such as the prostate gland PG, the prostatic urethra, and the prostate capsule boundary 2002. In some embodiments, a machine learning algorithm, such as a convolutional neural network (CNN), may be implemented to enhance image segmentation and identification of anatomical landmarks including the bladder neck 1202 and verumontanum VM. The anatomical model 1102 may identify potential sites for implant 10 placement, as shown in the planning guidance 1104 of FIG. 17 and FIG. 18, where numbered implant positions 1 through 6 and their trajectories are overlaid on the anatomical model 1102.

At operation 1906, an implant delivery device including at least one spatial sensor 110 is inserted into the body of the patient 1. For example, the elongate member 104 of the implant delivery device 100 (see FIG. 5) or the cartridge 204 of the implant delivery device 200 (see FIG. 6) may be inserted trans-urethrally through the urethra UT, e.g., as shown in FIG. 3A and FIG. 12. The spatial sensor 110 may be coupled to the handle assembly 102 or handle assembly 202, or may be enclosed within the elongate member 104, as described with respect to FIG. 8. In some embodiments, the spatial sensor 110 may comprise an inertial measurement unit (IMU) including accelerometers and gyroscopes, as described with respect to the system 900. In other embodiments, the spatial sensor 110 may comprise an electromagnetic (EM) tracking device configured to operate in conjunction with the field generator 1002 of the system 1000, as shown in FIG. 10. In some embodiments, the implant delivery device may include the sensor 2004 of the system 2000 as described with respect to FIGS. 20-22, which provides ultrasound-based distance measurements to the prostate capsule boundary 2002.

At operation 1908, the implant delivery device is aligned with the anatomy of the patient 1 based on the anatomical model 1102 and data received from the at least one spatial sensor 110. As shown for example in FIG. 12, the tip of the elongate member 104 may be inserted along the urethral axis 1204 up to the bladder neck 1202, which serves as a reference 1206 for calibrating or zeroing the location, position, and/or orientation of the implant delivery device 100 and/or implant delivery device 200. For example, a clinician may translationally position the elongate member 104 such that its distal tip contacts the bladder neck 1202, and may rotationally position the implant delivery device to a desired clock position (e.g., 3 o'clock or 9 o'clock) relative to the prostate gland PG. The clinician may then activate a user input of the system 900 and/or system 1000 to reset or calibrate the spatial sensor 110, thereby orienting the implant delivery device to the anatomical model 1102. In some embodiments, for example as described with respect to FIG. 11, the system 900 and/or system 1000 may automatically recognize the bladder neck 1202 based on images captured by the image sensor 912 and automatically align the implant delivery device with the anatomical model 1102. In some embodiments, as described with respect to FIGS. 20-22, the system 2000 may provide real-time feedback via the sensor 2004 to assist in aligning the implant delivery device at an optimal compression angle relative to the prostate capsule boundary 2002.

At operation 1910, at least one of a position, a location, or an orientation of at least a portion of the implant delivery device is tracked based on the data from the spatial sensor 110. As described with respect to FIG. 8, the spatial sensor 110 may be a 6-axis device that measures data relative to orthogonal axes aligned to a three-dimensional Cartesian coordinate system including the x-axis 802, y-axis 804, and z-axis 806. The accelerometers measure linear acceleration corresponding to forward/backward, left/right, and up/down movements, while the gyroscopes measure rotational rates capturing roll, pitch, and yaw motions. Collectively, these measurements provide six degrees of freedom (DOF), enabling the spatial sensor 110 to comprehensively capture the translational and rotational dynamics of the implant delivery device 100 and/or implant delivery device 200 in three-dimensional space. As shown in FIG. 13, the system 900 and/or system 1000 may track changes in the anatomy of patient 1 during the procedure, such as changes in the urethral axis 1204 compared to the anatomical model 1102. The tracking data may be used to generate real-time intraoperative guidance updates, as shown in the user interface 1400 of FIG. 14, FIG. 15, and FIG. 16, including the real-time device tip and compression angle 1502 displayed in FIG. 15. In some embodiments, as described with respect to FIGS. 20-22, the sensor 2004 of the system 2000 may supplement the spatial sensor 110 by providing distance measurements to the prostate capsule boundary 2002, enabling tracking of the shaft distal portion 2006 position relative to the prostatic capsule.

At operation 1912, an implant 10 is placed in the anatomy of the patient 1 via the implant delivery device. As shown in FIGS. 3A-3G, placing the implant 10 may include advancing a needle 32 through the distal anchor delivery device 30 to penetrate the tissue of the prostate gland PG until the distal end of the needle 32 emerges out of the prostate capsule boundary 2002 (see FIG. 3B and FIG. 3C). The distal anchor 12 connected to the connector 16 may be deployed at or just beyond the outer surface of the prostatic capsule (see FIG. 3C and FIG. 3D). The proximal anchor 14 may then be deployed on the inner wall of the urethra UT and attached to the connector 16 to complete the implant 10 (see FIG. 3F and FIG. 3G). The implant 10 may be deployed at an angle relative to the longitudinal axis of the urethra UT to target a specified lobe of the prostate gland PG, such as between the 1 o'clock and 3 o'clock position to target the left lateral lobe, or between the 9 o'clock and 11 o'clock position to target the right lateral lobe. As shown in FIG. 2, multiple implants 10 may be placed in the lateral lobes of the prostate gland PG to compress or retract regions of the prostate gland PG and relieve urethral constriction. In some embodiments, as described with respect to FIGS. 20-22, the system 2000 including the sensor 2004 may provide real-time feedback to guide needle 32 deployment depth and tissue compression, with the ultrasound sensor 2004 providing tissue structure gradient information and distance measurements to the prostate capsule boundary 2002 prior to needle 32 deployment to help properly and fully deploy the implant 10 and avoid bone strike.

With reference to FIGS. 20-22, a system for treating BPH is disclosed. The system 2000 is similar to and may include elements of other systems described herein, including system 900 and system 1000. The system 2000 may be a transurethral ultrasound system (TUUS). System 2000 is suitable for treating BPH and is configured for inserting an implant into the prostate. The tip of system 2000 is suitable for use with any system disclosed herein. The system 2000 includes a shaft distal portion 2006 of an elongate member 104 positioned within a urethra UT, and one or more sensors 2004 arranged along the shaft distal portion 2006. The system 2000 further includes a chip-on-tip (COT) imaging component 2008 coupled to the shaft distal portion 2006. The chip-on-tip imaging component 2008 comprises an image acquisition device, such as a camera or image sensor, located at or near the distal tip of the shaft distal portion 2006. The chip-on-tip imaging component 2008 enables visualization during device navigation and implant deployment by capturing images at the procedural site. In some embodiments, image data captured by the chip-on-tip imaging component 2008 may be processed locally at the tip, while in other embodiments, the image data may be transmitted to a separate processing unit to which the device connects for processing and display. FIG. 20 illustrates a side view of a system 2000 showing a shaft-mounted ultrasound configuration for measuring distance to a prostate capsule boundary 2002. The sensor 2004 comprises a Type-A ultrasound transducer mounted to the shaft distal portion 2006 and configured to emit ultrasound waves through prostatic tissue of the prostate gland PG toward the prostate capsule boundary 2002. The prostate capsule boundary 2002 is depicted as a curved boundary positioned below the shaft distal portion 2006 and the urethra UT. The Type-A ultrasound transducer is configured to determine the distance from the shaft distal portion 2006 to the prostate capsule boundary 2002 within a narrow or fixed vector corresponding to a needle 32 path, providing real-time feedback to guide needle 32 deployment depth and tissue compression during a BPH treatment procedure. In some embodiments, the sensor 2004 may also be configured to simultaneously detect a bone boundary to prevent the needle 32 from being deployed into pelvic or other bone, which could cause implant deployment to fail. The Type-A ultrasound transducer is simpler, smaller, and less costly than a full Type-B ultrasound array, making it suitable for integration into a single-use delivery device. A Type B ultrasound array may be used as well, if desired. The chip-on-tip imaging component 2008 may be integrated with additional features such as an inertial measurement unit (IMU) comprising accelerometers and gyroscopes to provide data on linear and rotational movement of the shaft distal portion 2006, pressure sensors configured to detect tissue contact and compression forces, temperature sensors, and signal conditioning circuitry. In some embodiments, the chip-on-tip imaging component 2008 may provide imaging capabilities that generate visual feedback to assist in navigating the device to the proper location within the prostate gland PG. With the help of the chip-on-tip imaging component 2008, the device can be easily navigated to the proper location, and the chip-on-tip imaging component 2008 can help navigate the needle 32 and provide visual feedback to help deploy the implant. The integration of the chip-on-tip imaging component 2008 with the sensor 2004 enables the device to be easily navigated to the proper location within the prostate gland PG, while the ultrasound sensor 2004 provides tissue structure gradient information and distance measurements to the prostate capsule boundary 2002 prior to needle 32 deployment, helping to properly and fully deploy the implant and avoid bone strike.

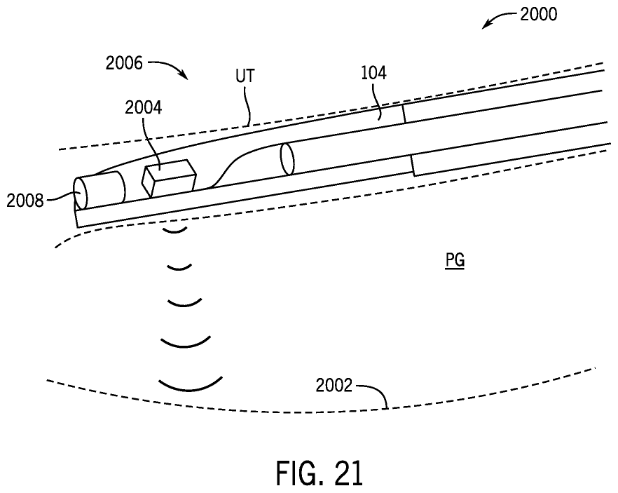

FIG. 21 illustrates a step of using the system 2000 wherein a user is guided to place the elongate member 104 at a compression angle that compresses the prostate gland PG and optimizes the distance between the shaft distal portion 2006 and the prostate capsule boundary 2002 for deploying one or more implants 10. The system 2000 provides feedback to assist the clinician in achieving proper tissue compression prior to needle deployment. The sensor 2004 may be used in conjunction with the display 2306 to provide real-time visual feedback indicating the position and orientation of the elongate member 104 relative to the prostate capsule boundary 2002 and the target deployment site. The chip-on-tip imaging component 2008 processes data from the sensor 2004 and any integrated IMU or pressure sensors locally at the shaft distal portion 2006, reducing latency between sensor measurement and feedback generation, which improves the accuracy of device placement by enabling more responsive guidance during positioning adjustments. In some embodiments, auditory cues may be generated to alert the user when the elongate member 104 approaches or reaches the desired compression angle and position for implant deployment. For example, the auditory cues may include spoken instructions or tones that change in frequency or intensity as the elongate member 104 approaches the optimal position, or distinct sounds that indicate when the correct positioning has been achieved. The system 2000 may operate in a closed-loop fashion, wherein sensor data from the sensor 2004 is continuously processed by the chip-on-tip imaging component 2008 and feedback is provided to the user via the display 2306 and/or auditory signals, enabling iterative adjustments until the elongate member 104 is correctly positioned for deployment of the implant. The closed-loop system provides feedback to assist the clinician in achieving proper tissue compression prior to needle deployment, with the display 2306 presenting visual indicators such as distance measurements, angle readouts, or graphical representations of the elongate member 104 position relative to anatomical landmarks, while auditory cues supplement the visual feedback to enhance situational awareness during the procedure.

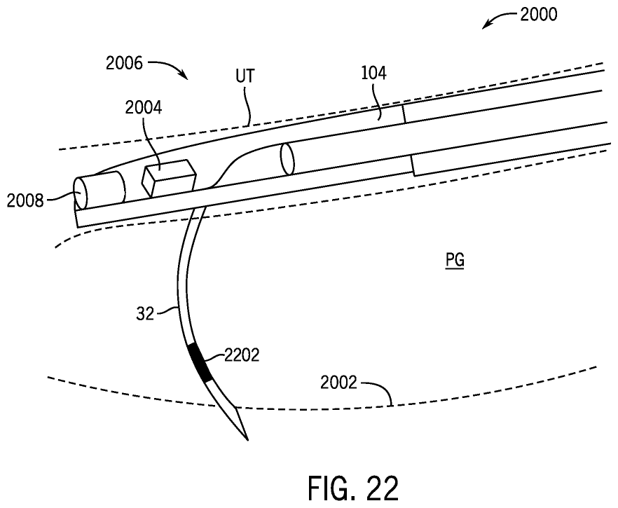

FIG. 22 illustrates a subsequent step of using the system 2000 wherein the needle 32 is deployed in the optimized position. The needle 32 is fired at an optimal compression angle ensuring piercing of the prostate capsule boundary 2002 while avoiding bone strike. The needle 32 trajectory achieves proper penetration depth through the prostatic tissue of the prostate gland PG to reach the outer surface of the prostatic capsule without extending beyond into surrounding anatomical structures such as pelvic bone. In some embodiments, a two-step compression may be performed, wherein a first level of compression is performed prior to needle deployment guided by the sensor 2004, the needle 32 is deployed, and then a second level of compression is performed with the needle 32 deployed. The required extent of the second level of compression may be calculated from the ultrasound input prior to deployment, and the compression itself may be guided by a spatial sensor 110 when the deployed needle 32 renders the ultrasound inoperable. The shaft distal portion 2006 may include an isolation window of anechoic material within which the sensor 2004 is embedded. The procedural space may be irrigated and submerged in water to promote ultrasound coupling between the sensor 2004 and the prostatic tissue. In some embodiments, the needle 32 tip may further comprise a sensor 2202 such as an impedance sensor or other contact sensor configured to detect when the needle 32 has pierced the prostate capsule boundary 2002. The sensor 2202 may detect piercing of the prostate capsule boundary 2002 based on a change in impedance or other physical parameter resulting from the transition between prostatic tissue and the tissue or space beyond the prostatic capsule.

Referring to FIGS. 20-22, the system 2000 provides an optimized approach for ensuring successful needle deployment through the prostate capsule boundary 2002 while avoiding pelvic bone strike. The system 2000 addresses a fundamental challenge in BPH treatment procedures: confirming that the needle 32 will successfully penetrate the prostate capsule boundary 2002 at an appropriate depth. If the needle 32 is deployed too far, there is a risk of striking pelvic bone, and if the needle 32 is not deployed far enough, the implant may lack sufficient capsule surface engagement, either of which can result in a failed deployment.

The system 2000 offers advantages over alternative approaches to solving this problem. While 3D reconstruction from TRUS or MRI imaging as described with respect to the anatomical model 1102 can provide information about the prostate capsule boundary 2002, such reconstruction has inherent limitations. Opportunities for error exist in image segmentation and registration with handle motion and position, particularly depending on the placement of the calibration point at the bladder neck 1202. Additionally, if the implant delivery device moves during the procedure, it can deform the anatomical model 1102, reducing accuracy.

Live TRUS imaging as described with respect to TRUS 1604 provides real-time feedback on device position and capsule boundary location, addressing some limitations of pre-operative reconstruction. However, significant barriers exist to user adoption of TRUS, as it is not part of the standard workflow, is uncomfortable for patients, and interferes with device maneuverability. A TRUS probe is a Type-B ultrasound that produces 2D or 3D images using an array of ultrasound elements, adding cost and complexity.

The system 2000 recognizes that the actual need is not to visualize the entire prostate, but rather to have confidence that the needle 32 tip, which has a predictable and localized position when deployed, will successfully penetrate the prostate capsule boundary 2002. The Type-A ultrasound transducer of the sensor 2004 is optimized for simplicity and cost, measuring distance to the prostate capsule boundary 2002 within a narrow or fixed vector corresponding to the needle 32 path. This targeted approach provides the necessary information for successful deployment without the complexity, cost, and workflow disruption associated with full Type-B ultrasound arrays. Preexisting systems necessitate needle deployment to be performed without real-time capsule distance feedback, relying on training guidance to compress the tissue with a certain degree of angulation applied to the handle. The system 2000 replaces this approach with real-time, localized feedback specific to the needle deployment vector.

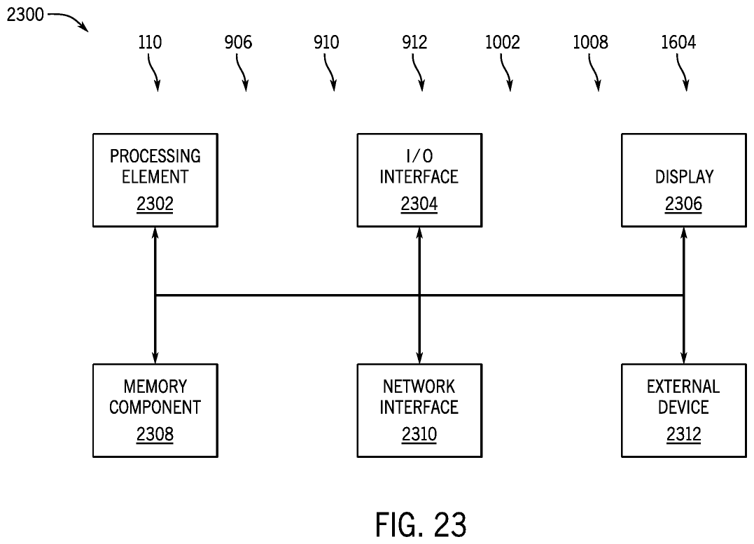

FIG. 23 is a simplified block diagram of components of a computing system 2300 of the system 900, the system 1000, and/or components thereof. For example, any of the spatial sensor 110, the image processing unit 906, the image sensor 912, the field generator 1002, the tracking control unit 1008, and/or the TRUS 1604 may include a computing system 2300. In any computing system 2300, the processing element 2302 and the memory component 2308 may be located at one or in several computing systems 2300. This disclosure contemplates any suitable number of such computing systems 2300. For example, the image processing unit 906 may be part of a desktop computing system, a mainframe, a blade, a mesh of computing systems 2300, a laptop or notebook computing system 2300, a tablet computing system 2300, an embedded computing system 2300, a system-on-chip, a single-board computing system 2300, or a combination of two or more of these. Where appropriate, a computing system 2300 may include one or more computing systems 2300; be unitary or distributed; span multiple locations; span multiple machines; span multiple data centers; or reside in a cloud, which may include one or more cloud components in one or more networks. A computing system 2300 may include one or more processing elements 2302, an input/output I/O interface 2304, one or more external devices 2312, one or more memory components 2308, and a network interface 2310. Each of the various components may be in communication with one another through one or more buses or communication networks, such as wired or wireless networks. The components in FIG. 23 are exemplary only. In various examples, the computing system 2300 may include additional components and/or functionality not shown in FIG. 23.

The processing element 2302 may be any type of electronic device capable of processing, receiving, and/or transmitting instructions. For example, the processing element 2302 may be a central processing unit, microprocessor, processor, or microcontroller. Additionally, it should be noted that some components of the computing system 2300 may be controlled by a first processing element 2302 and other components may be controlled by a second processing element 2302, where the first and second processing elements may or may not be in communication with each other.

The I/O interface 2304 allows a user to enter data in to computing system 2300, as well as provides an input/output for the computing system 2300 to communicate with other devices or services. The I/O interface 2304 can include one or more input buttons, touch pads, touch screens, and so on.

The external device 2312 are one or more devices that can be used to provide various inputs to the computing systems 2300, e.g., mouse, microphone, keyboard, trackpad, sensing element (e.g., a thermistor, humidity sensor, light detector, etc. The external devices 2312 may be local or remote and may vary as desired. In some examples, the external devices 2312 may also include one or more additional sensors.

The memory components 2308 are used by the computing system 2300 to store instructions for the processing element 2302 such as for executing the methods disclosed herein, as well as store an AI algorithm, the training data for such, intra-operative imaging data, planning data, the anatomical model 1102, user preferences, alerts, etc. The memory components 2308 may be, for example, magneto-optical storage, read-only memory, random access memory, erasable programmable memory, flash memory, or a combination of one or more types of memory components.

The network interface 2310 provides communication to and from the computing system 2300 to other devices. The network interface 2310 includes one or more communication protocols, such as, but not limited to Wi-Fi, Ethernet, Bluetooth, etc. The network interface 2310 may also include one or more hardwired components, such as a Universal Serial Bus (USB) cable, or the like. The configuration of the network interface 2310 depends on the types of communication desired and may be modified to communicate via Wi-Fi, Bluetooth, etc.

The display 2306 provides a visual output for the computing system 2300 and may be varied as needed based on the device. The display 2306 may be configured to provide visual feedback to the user, such as a clinician and may include a liquid crystal display screen, light emitting diode screen, plasma screen, or the like. In some examples, the display 2306 may be configured to act as an input element for the clinician through touch feedback or the like.

The description of certain embodiments included herein is merely exemplary in nature and is in no way intended to limit the scope of the disclosure or its applications or uses. In the included detailed description of embodiments of the present systems and methods, reference is made to the accompanying drawings which form a part hereof, and which are shown by way of illustration specific to embodiments in which the described systems and methods may be practiced. These embodiments are described in sufficient detail to enable those skilled in the art to practice presently disclosed systems and methods, and it is to be understood that other embodiments may be utilized, and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The included detailed description is therefore not to be taken in a limiting sense, and the scope of the disclosure is defined only by the appended claims.

From the foregoing it will be appreciated that, although specific embodiments of the invention have been described herein for purposes of illustration, various modifications and combinations may be made without deviating from the spirit and scope of the invention.

The particulars shown herein are by way of example and for purposes of illustrative discussion of the preferred embodiments of the present disclosure and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. The following embodiments are hereby disclosed and incorporated into the Detailed Description, with each embodiment standing on its own or combinable with any other embodiment:

In an embodiment, a method of treating a prostate gland involves generating a plurality of images of an anatomy of a patient; based on the plurality of images, generating an anatomical model of the anatomy; inserting, into the patient, an elongate member or portion of an implant delivery device, wherein the implant delivery device includes at least one spatial sensor; aligning the elongate member or portion of the implant delivery device with the anatomy based on the anatomical model and data received from the at least one spatial sensor; tracking at least one of a position, a location, or an orientation of at least a portion of the implant delivery device based on the data; and placing an implant in the anatomy via the implant delivery device.

In an embodiment the plurality of images includes at least one image selected from the group consisting of MRI images, CT images, and transrectal ultrasound images. This embodiment enables generation of high-resolution cross-sectional images that can be integrated and assembled into a three-dimensional representation, providing comprehensive volumetric data of the patient's anatomy for accurate treatment planning.

In an embodiment the anatomical model includes one or more representations of anatomical structures comprising the prostate gland and/or the prostatic urethra, and identifies potential sites for implant placement. This embodiment facilitates pre-surgical planning by isolating specific structures or regions of interest and identifying optimal implant locations, thereby improving the precision of implant deployment.

In an embodiment the anatomical model is generated using a machine learning algorithm that enhances image segmentation and identification of anatomical landmarks. This embodiment improves the accuracy of anatomical structure differentiation by interpreting grayscale values or signals corresponding to different tissue densities or properties, enabling creation of a more detailed and interactive anatomical model compared to other techniques.

In an embodiment aligning the implant delivery device comprises translationally and/or rotationally positioning the elongate member or portion of the implant delivery device relative to a reference portion of the anatomy. This embodiment enables precise calibration of the delivery device position and orientation, reducing accumulated error over time and improving the accuracy of subsequent tracking during the procedure compared to other techniques.

In an embodiment the reference portion is a bladder neck. This embodiment provides a consistent and identifiable anatomical landmark for zeroing, resetting, or calibrating the location, position, and/or orientation of the implant delivery device, enabling reliable alignment with the anatomical model.

In an embodiment the aligning step includes using data from the spatial sensor to assist in the alignment. This embodiment enables real-time feedback during the alignment process, allowing the clinician to make precise adjustments and orient the implant delivery device to the anatomical model with improved accuracy compared to other techniques.

In an embodiment the method further involves generating real-time intraoperative guidance updates based on the data from the at least one spatial sensor to aid in accurate implant navigation and placement. This embodiment enables the clinician to adjust for movements or changes in the anatomy of the patient, differences discovered between the anatomical model and the actual anatomy, or failure of an implant to be properly placed, thereby improving patient outcomes compared to other techniques.

In an embodiment the intraoperative guidance updates include visualization outputs that display real-time movement of the elongate member or portion of the implant delivery device in conjunction with the anatomical model. This embodiment provides visual indicators such as distance measurements, angle readouts, or graphical representations of the elongate member position relative to anatomical landmarks, enhancing situational awareness for the clinician during the procedure.

In an embodiment the tracking of the implant delivery device is based on a combination of accelerometer and gyroscope data from the spatial sensor. This embodiment enables comprehensive capture of both linear acceleration and rotational rates, providing motion data that can be processed to derive the current acceleration, velocity, position, and/or orientation of the implant delivery device.

In an embodiment the data includes six degrees of freedom. This embodiment enables the spatial sensor to comprehensively capture the translational and rotational dynamics of the implant delivery device in three-dimensional space, providing complete tracking of device movement during the procedure.

In an embodiment placing the implant includes deploying the implant at an angle relative to a longitudinal axis of the urethra to target a specified lobe of the prostate gland. This embodiment enables targeted treatment of specific lobes of the prostate gland, such as the left lateral lobe, right lateral lobe, or middle lobe, allowing for customized treatment based on the patient's anatomy.

In an embodiment the implant delivery device includes a sensor mounted to a shaft distal portion of the elongate member, the sensor configured to measure a distance from the shaft distal portion to a prostate capsule boundary. This embodiment provides real-time feedback to guide needle deployment depth and tissue compression during the procedure, enabling confirmation that the needle will successfully penetrate the prostate capsule boundary at an appropriate depth.

In an embodiment the sensor comprises a Type-A ultrasound transducer configured to emit ultrasound waves through prostatic tissue toward the prostate capsule boundary. This embodiment provides a simpler, smaller, and less costly solution than a Type-B ultrasound array, making it suitable for integration into a single-use delivery device while still providing the necessary distance measurement information for successful deployment.

In an embodiment the method further involves guiding a user to place the elongate member at a compression angle that compresses the prostate gland and optimizes a distance to the prostate capsule boundary prior to deploying a needle through the prostatic tissue. This embodiment assists the clinician in achieving proper tissue compression prior to needle deployment, optimizing the distance between the shaft distal portion and the prostate capsule boundary for deploying implants.

In an embodiment deploying the needle comprises firing the needle at the compression angle to pierce the prostate capsule boundary while avoiding bone strike. This embodiment ensures proper penetration depth through the prostatic tissue to reach the outer surface of the prostatic capsule without extending beyond into surrounding anatomical structures such as pelvic bone, preventing failed deployments.

In an embodiment, a delivery device is configured to deploy one or more implants to a prostate gland of a patient. The delivery device includes a handle assembly and an elongate shaft assembly; and a sensor coupled to the handle assembly and configured to locate a portion of the elongate shaft assembly with respect to anatomy of the patient. This embodiment enables real-time tracking of the delivery device position relative to the patient's anatomy, improving the accuracy of implant placement compared to prior delivery devices that lack spatial sensing capabilities.

In an embodiment the sensor is an inertial measurement unit (IMU) that includes accelerometers and gyroscopes to provide data on linear and rotational movement of the elongate shaft assembly. This embodiment provides comprehensive data on movement, position, location, and/or orientation of the implant delivery device through measurement of specific force, angular rate, and real-time tracking of such data.

In an embodiment the delivery device further includes an ultrasound transducer mounted to a distal portion of the elongate shaft assembly and configured to measure a distance from the distal portion to a prostate capsule boundary. This embodiment provides tissue structure gradient information and distance measurements to the prostate capsule boundary prior to needle deployment, helping to properly and fully deploy the implant and avoid a bone strike.

In an embodiment the ultrasound transducer is a Type-A ultrasound transducer configured to determine the distance within a narrow or fixed vector corresponding to a needle path. This embodiment provides targeted distance measurement specific to the needle deployment vector, replacing the prior approach of relying on training guidance to compress the tissue with a certain degree of angulation applied to the handle, with real-time, localized feedback.

In an embodiment, a system for treating a prostate gland of the patient includes the delivery device of Example 17; an imaging device configured to capture images of the anatomy of the patient; and a processing element configured to receive the images of the anatomy of the patient, and generate a three dimensional model of the anatomy. This embodiment enables creation of a detailed, interactive three-dimensional anatomical model that can be used for diagnostic purposes, pre-surgical planning, and intra-operative guidance, thereby improving the placement of implants and overall procedural outcome compared to other techniques.

In an embodiment the system further includes an inertial measurement unit (IMU) and a display, the display being configured to present real-time feedback via a user interface about the position and orientation of the elongate shaft assembly relative to the anatomy based on data from the IMU. This embodiment provides visual feedback to the clinician during the procedure, enabling iterative adjustments until the elongate member is correctly positioned for deployment of the implant.

In an embodiment the delivery device further comprises an ultrasound transducer mounted to a distal portion of the elongate shaft assembly and configured to measure a distance to a prostate capsule boundary. This embodiment addresses a challenge in BPH treatment procedures of confirming that the needle will successfully penetrate the prostate capsule boundary at an appropriate depth, reducing the risk of failed deployments compared to other techniques.

In an embodiment, a system for delivering and deploying implants to a prostate gland of a patient includes an implant delivery device including an elongate shaft assembly; a spatial sensor coupled to the implant delivery device and configured to provide data on a position and/or orientation of the elongate shaft assembly relative to anatomy of the patient; a processing unit configured to generate an anatomical model of the anatomy from preoperative imaging data; and a display configured to provide intraoperative guidance by integrating the spatial sensor data with the anatomical model. This embodiment provides improved patient outcomes compared to prior methods and systems of treating BPH by enabling real-time tracking and guidance during the procedure through integration of spatial sensor data with preoperative anatomical modeling.

In an embodiment the spatial sensor is an inertial measurement unit (IMU) comprising accelerometers and gyroscopes to track linear and/or rotational movement of the elongate shaft assembly. This embodiment enables measurement of linear acceleration and rotational rates about orthogonal axes, providing six degrees of freedom for comprehensive tracking of the delivery device trajectory, attitude, position, location, and/or orientation.

In an embodiment the spatial sensor is an electromagnetic tracking device working in coordination with a field generator to track linear and/or rotational movement of the elongate shaft assembly. This embodiment enables precise monitoring of instrument positions and orientations relative to the patient's anatomy by detecting disturbances within a controlled electromagnetic field as the implant delivery device moves through the field.

In an embodiment the field generator creates a controlled electromagnetic field covering a surgical area. This embodiment provides a defined field range within which the spatial sensor can detect disturbances, enabling continuous updating and processing of data to provide a dynamic and accurate depiction of position, location, and/or orientation of surgical instruments in real-time.

In an embodiment the processing unit is configured to provide real-time intraoperative updates of the elongate shaft assembly position and/or orientation on the display. This embodiment enables the clinician to adjust for movements or changes in the anatomy during the procedure, such as changes in the urethral axis compared to the anatomical model, improving procedural accuracy compared to other techniques.

In an embodiment the display presents preoperative planning outputs and intraoperative guidance simultaneously. This embodiment enables the clinician to view both the planned implant locations and real-time device position in a unified interface, facilitating comparison between the treatment plan and actual procedure progress.

In an embodiment the system further includes a transrectal ultrasound unit that supplements anatomical visualization during the procedure. This embodiment provides live intraoperative imaging in addition to images captured by the image sensor, enabling real-time visualization of the prostate and surrounding structures during implant deployment.

In an embodiment the processing unit utilizes a machine learning algorithm to refine the anatomical model based on intraoperative data of previous cases. This embodiment enables continual improvement of the anatomical model, planning guidance, and intraoperative guidance through training data generated from prior procedures, providing predictive insights into alternative surgical strategies and outcomes.

In an embodiment the implant delivery device further comprises an ultrasound transducer mounted to a distal portion of the elongate shaft assembly and configured to measure a distance from the distal portion to a prostate capsule boundary. This embodiment provides real-time, localized feedback specific to the needle deployment vector, replacing the prior approach of performing needle deployment without real-time capsule distance feedback.

In an embodiment the ultrasound transducer is a Type-A ultrasound transducer configured to emit ultrasound waves through prostatic tissue toward the prostate capsule boundary. This embodiment provides the necessary information for successful deployment without the complexity, cost, and workflow disruption associated with Type-B ultrasound arrays, while being suitable for integration into a single-use delivery device.

In an embodiment the display is configured to provide real-time visual feedback indicating a position and orientation of the elongate shaft assembly relative to a prostate capsule boundary and a target deployment site. This embodiment enables more responsive guidance during positioning adjustments compared to other techniques by reducing latency between sensor measurement and feedback generation, improving the accuracy of device placement.

In an embodiment the implant delivery device further comprises a chip-on-tip imaging component mounted to the distal portion of the elongate shaft assembly and configured to provide visual feedback for navigating the implant delivery device to a target location within the prostate gland. This embodiment enables the device to be easily navigated to the proper location within the prostate gland by providing imaging capabilities that generate visual feedback to assist in device navigation and implant deployment.

In an embodiment the ultrasound transducer and the chip-on-tip imaging component are integrated on a single shaft to provide combined imaging capabilities during the procedure. This embodiment provides combined imaging by integrating ultrasound for tissue structure gradient and distance measurement with chip-on-tip imaging for visual navigation, enabling both proper device navigation and confirmation of appropriate needle deployment depth.

In an embodiment the chip-on-tip imaging component is configured to process image data locally at the distal portion of the elongate shaft assembly and provide real-time visual guidance during device navigation and implant deployment. This embodiment reduces latency between sensor measurement and feedback generation by processing data locally at the shaft distal portion, improving the accuracy of device placement by enabling more responsive guidance during positioning adjustments.

In an embodiment a location or arrangement of the ultrasound transducer on the distal portion of the elongate shaft assembly is configurable. This embodiment enables optimization of the ultrasound transducer position to accommodate different anatomical configurations and procedural requirements, providing flexibility in device design and deployment approach.

In an embodiment a location or arrangement of the ultrasound transducer and the chip-on-tip imaging component on the distal portion of the elongate shaft assembly is configurable. This embodiment enables optimization of both imaging components to accommodate different anatomical configurations and procedural requirements, providing flexibility in achieving combined imaging capabilities for various clinical scenarios.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

All relative, directional, and ordinal references (including top, bottom, side, front, rear, first, second, third, and so forth) are given by way of example to aid understanding of the reader of the examples described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other, unless specifically set forth in the claims.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. A delivery device configured to deploy one or more implants to a prostate gland of a patient, comprising:
a handle assembly and an elongate shaft assembly; and
a sensor coupled to the handle assembly and configured to locate a portion of the elongate shaft assembly with respect to anatomy of the patient.

2. The delivery device of claim 1, wherein the sensor is an inertial measurement unit (IMU) that includes accelerometers and gyroscopes to provide data on linear and rotational movement of the elongate shaft assembly.

3. The delivery device of claim 1, further comprising an ultrasound transducer mounted to a distal portion of the elongate shaft assembly and configured to measure a distance from the distal portion to a prostate capsule boundary.

4. The delivery device of claim 3, wherein the ultrasound transducer is a Type-A ultrasound transducer configured to determine the distance within a narrow or fixed vector corresponding to a needle path.

5. A system for treating a prostate gland of the patient comprising:
the delivery device of claim 1;
an imaging device configured to capture images of the anatomy of the patient; and
a processing element configured to:
receive the images of the anatomy of the patient, and
generate a three dimensional model of the anatomy.

6. The system of claim 5, further comprising an inertial measurement unit (IMU) and a display, the display being configured to present real-time feedback via a user interface about the position and orientation of the elongate shaft assembly relative to the anatomy based on data from the IMU.

7. The system of claim 5, wherein the delivery device further comprises an ultrasound transducer mounted to a distal portion of the elongate shaft assembly and configured to measure a distance to a prostate capsule boundary.

8. A system for delivering and deploying implants to a prostate gland of a patient, comprising:
an implant delivery device including an elongate shaft assembly;
a spatial sensor coupled to the implant delivery device and configured to provide data on a position and/or orientation of the elongate shaft assembly relative to anatomy of the patient;
a processing unit configured to generate an anatomical model of the anatomy from preoperative imaging data; and
a display configured to provide intraoperative guidance by integrating the spatial sensor data with the anatomical model.

9. The system of claim 8, wherein the spatial sensor is an inertial measurement unit (IMU) comprising accelerometers and gyroscopes to track linear and/or rotational movement of the elongate shaft assembly.

10. The system of claim 8, wherein the spatial sensor is an electromagnetic tracking device working in coordination with a field generator to track linear and/or rotational movement of the elongate shaft assembly, wherein the field generator creates a controlled electromagnetic field covering a surgical area.

11. The system of claim 8, wherein the processing unit is configured to provide real-time intraoperative updates of the elongate shaft assembly position and/or orientation on the display.

12. The system of claim 8, wherein the display presents preoperative planning outputs and intraoperative guidance simultaneously.

13. The system of claim 8, wherein the processing unit utilizes a machine learning algorithm to refine the anatomical model based on intraoperative data of previous cases.

14. The system of claim 8, wherein the implant delivery device further comprises an ultrasound transducer mounted to a distal portion of the elongate shaft assembly and configured to measure a distance from the distal portion to a prostate capsule boundary.

15. The system of claim 14, wherein the ultrasound transducer is a Type-A ultrasound transducer configured to emit ultrasound waves through prostatic tissue toward the prostate capsule boundary.

16. The system of claim 8, wherein the display is configured to provide real-time visual feedback indicating a position and orientation of the elongate shaft assembly relative to a prostate capsule boundary and a target deployment site.

17. The system of claim 14, wherein the implant delivery device further comprises a chip-on-tip imaging component mounted to the distal portion of the elongate shaft assembly and configured to provide visual feedback for navigating the implant delivery device to a target location within the prostate gland.

18. The system of claim 17, wherein the ultrasound transducer and the chip-on-tip imaging component are integrated on a single shaft to provide combined imaging capabilities during a procedure.

19. The system of claim 17, wherein the chip-on-tip imaging component is configured to process image data locally at the distal portion of the elongate shaft assembly and provide real-time visual guidance during device navigation and implant deployment, and wherein a location or arrangement of the ultrasound transducer and the chip-on-tip imaging component on the distal portion of the elongate shaft assembly is configurable.

20. The system of claim 14, wherein a location or arrangement of the ultrasound transducer on the distal portion of the elongate shaft assembly is configurable.
